(19) United States
(12) Patent Application Publication
(10) Pub. No.: US 2010/0150911 A1
(43) Pub. Date: Jun. 17, 2010

(54) METHODS AND SYSTEMS OF TREATING AGE-RELATED MACULAR-DEGENERATION

(75) Inventors: Jarbas Caiado De Castro Neto, Sao Paulo (BR); Mario Antonio Stefani, Sao Paulo (BR); Giuliano Rossi, Sao Paulo (BR); Alessandro Damiani Mota, Sao Paulo (BR); Rogerio Alves Costa, Sao Paulo (BR); Jose Augusto Cardillo, Sao Paulo (BR); Jairo Kerr Azevedo, South Australia (AU)

Correspondence Address: JONES DAY, 222 EAST 41ST ST, NEW YORK, NY, 10017, US

(73) Assignee: OPTO GLOBAL HOLDINGS PTY LTD, South Australia (AU)

(21) Appl. No.: 12/449,528

(22) PCT Filed: Feb. 14, 2008
(86) PCT No.: PCT/AU2008/000191

§ 371 Date: Jan. 29, 2010

Related U.S. Application Data
(60) Provisional application No. 60/901,068, filed on Feb. 14, 2007.
(60) Provisional application No. 61/004,768, filed on Nov. 30, 2007.

(51) Int. Cl. A61K 39/395; A61K 31/58; A61K 31/57; A61K 31/501

(52) U.S. Cl. 424/133.1; 424/158.1; 514/174; 514/179; 514/252.03

(57) ABSTRACT

A method of treating CNV secondary to AMD, comprising administering a medicament in combination with i-MP treatment where the i-MP treatment includes a control method comprising the following steps: determining and/or inputting at least one dosage parameter dependent on patient-related data; determining and/or inputting at least one application parameter dependent on patient-related data; providing instructions to administer ICG to the patient; and providing instructions to apply an output of an application device to a treatment area of the patient; where at least one of the steps is a computer-implemented step.

### INCORPORATION BY REFERENCE

The present application is related to U.S. Provisional Application No. 60/901,068, filed on Feb. 14, 2007, U.S. Provisional Application No. 61/004,768, filed on Nov. 30, 2007, International Application No. PCT/AU2006/001147, filed on Aug. 11, 2006, International Application No. PCT/AU2006/000721 (WO 06/125280), filed on May 29, 2006, International Application No. PCT/BR02/00010 (WO 02/094260), filed on Jan. 22, 2002, Australian Provisional Application No. AU2005904315, filed on Aug. 11, 2005, Australian Provisional Application No. AU2005902720, filed on May 27, 2005, Brazilian application No. PI 0102052-8, filed on May 21, 2001, and U.S. Provisional Application No. 60/901,068 filed Feb. 14, 2007. Each of these applications is herein incorporated by reference in its entirety.

### FIELD OF THE INVENTION

This invention relates to treatment of Age-related Macular Degeneration (AMD) and more specifically relates to combination therapies for treatment of AMD.

### BACKGROUND OF THE INVENTION

Age-related Macular Degeneration (AMD) is the leading cause of legal blindness in people over 60 years old in the developed countries. The neovascular (exudative) form of AMD is associated with abnormal blood vessel growth known as neovascularization from the choroid. AMD is the result of abnormal blood vessel growth known as neovascularization in the retina from the choroid. This growth, known as CNV (choroidal neovascularization) results in weak new blood vessels that leak fluid into the retina causing damage and scarring and ultimately loss of visual acuity and severe irreversible vision loss.

Conventional treatments, either device based therapies like macular photocoagulation and photodynamic therapy or drug therapies, exhibit disadvantages such as complexity of treatment, vision loss, limited treatment populations, high retreatment ratio and high cost. In particular, existing drug therapies have an extremely high cost per treatment and high frequency (monthly) of treatment. Each of these limitations also results in reduced patient compliance, increased patient inconvenience, increased risk of endophthalmitis and increased likelihood of medication error.

For example, treatment of AMD ranibizumab (LUCENTIS) requires monthly injections, while pegaptanib sodium (MACUGEN) treatment requires injections every 6 weeks and bevacizumab (AVASTIN) requires injections every 6 to 8 weeks, presumably. These frequent injection regimens include high costs of the drug, risk of endophthalmitis and other complications, patient inconvenience associated with arranging transportation to the treatment clinic, patient discomfort with regular perpetual injections into the eye, limitation of treatment availability as a result of saturation of available treating physician time and capabilities and potential increased risk of stroke at 2 years after commencement of treatment

The present invention combines drug therapy with indocyanine green-mediated photothrombosis (i-MP) therapy and reduces the frequency of treatment, patient inconvenience, cost, risk of endophthalmitis and likelihood of medication error and while improving patient compliance and treatment outcomes.

### BRIEF SUMMARY OF THE INVENTION

The invention provides methods of treating choroidal neovascularization (CNV), including classic, occult and mixed types, that invade the subretinal space or neurosensory tissue resulting from, for example, AMD, pathological myopia, angioid streaks, syndrome of presumed ocular histoplasmosis, central serous retinopathy, idiopathic polypoidal choroidal vasculopathy, and other conditions resulting from inflammatory and idiopathic causes, by administering to a patient in need thereof at least one medicament in combination with indocyanine green (ICG)-mediated photothrombosis (i-MP) treatment. The i-MP treatment used may vary with certain embodiments (for example, but not limited to, the procedures disclosed in PCT/AU2006/001147, PCT/AU2006/000721 (WO 06/125280), PCT/BR02/00010 (WO 02/094260), Australian Provisional Application No. AU2005904315, Australian Provisional Application No. AU2005902720, Brazilian application No. PI 0102052-8, U.S. Provisional Application No. 60/901,068, other i-MP procedures disclosed herein, or combinations thereof.

In some embodiments, the medicament may be selected from the group consisting of antiangiogenesis compounds, antiproliferative compounds, cytotoxic compounds, immunomodulators, anti-inflammatory compounds and combinations thereof.

In some embodiments, the medicament is not an anti-inflammatory or steroid based-compound, for example in some embodiments, the medicament is not triamcinolone or triamcinolone acetonide. In some embodiments, the invention comprises a method of treating CNV secondary to AMD, comprising administering a medicament in combination with i-MP treatment, where the medicament is not an anti-inflammatory compound. The i-MP treatment used may vary with certain embodiments (for example, but not limited to, the procedures disclosed in PCT/AU2006/001147, PCT/AU2006/000721 (WO 06/125280), PCT/BR02/00010 (WO 02/094260), Australian Provisional Application No. AU2005904315, Australian Provisional Application No. AU2005902720, Brazilian application No. PI 0102052-8, U.S. Provisional Application No. 60/901,068, other i-MP procedures disclosed herein, or combinations thereof.

In some embodiments, the invention includes a method of treating CNV secondary to AMD, comprising administering a medicament in combination with i-MP treatment, where the i-MP treatment includes a control method comprising the following steps:

determining and/or inputting at least one dosage parameter dependent on patient-related data;

determining and/or inputting at least one application parameter dependent on patient-related data;

providing instructions to administer ICG to the patient; and

providing instructions to apply an output of an application device to a treatment area of the patient; where at least one of the steps is a computer-implemented step.

In some embodiments, a method of treating CNV secondary to AMI) includes administering to a patient in need thereof, one or more antiangiogenesis compounds in combination with i-MP treatment, where the i-MP treatment includes a control method comprising the following steps:

determining and/or inputting at least one dosage parameter dependent on patient-related data;

determining and/or inputting at least one application parameter dependent on patient-related data;

providing instructions to administer ICG to the patient; and

providing instructions to apply an output of an application device to a treatment area of the patient; where at least one of the steps is a computer-implemented step.

In some embodiments a method of treating CNV secondary to AMD, includes administering to a patient in need thereof, one or more antiangiogenesis compounds in combination with i-MP treatment, where the i-MP treatment includes a control method comprising the following steps:

determining and/or inputting at least one dosage parameter dependent on patient-related data;

determining and/or inputting at least one application parameter dependent on patient-related data;

providing instructions to administer ICG to the patient; and

providing instructions to apply an output of an application device to a treatment area of the patient; where at least one of the steps is a computer-implemented step, where the i-MP treatment is performed on the patient up to 3 weeks before and/or after administration of the antiangiogenesis compound.

In some embodiments, the antiangiogenesis compound may be an anti-VEGF (Vascular Endothelial Growth Factor) compound, in that it inhibits VEGF-promoted/mediated angiogenesis. In other embodiments, the antiangiogenesis compound may be a steroidal or steroid-based compound, such as a mineralocorticoid or glucocorticoid-based compound. In yet other embodiments, the antiangiogenesis compound may be an anti-HIF (Hypoxia Inducible Factor) compound, such as an anti-HIF1α or anti-HIF2α compound and may inhibit HIF-promoted/mediated angiogenesis. In other embodiments, the antiangiogenesis compound may be an anti-FGF (Fibroblast Growth Factor) compound, such as an anti-FGF2 compound and may inhibit FGF-promoted/mediated angiogenesis. In still other embodiments, the antiangiogenesis compound may be an anti-HGF (Hepatocyte Growth Factor) compound may inhibit HGF-promoted/mediated angiogenesis

In some embodiments, a method of treating CNV secondary to AMD includes administering to a patient in need thereof, one or more antiproliferative compounds in combination with i-MP treatment, where the i-MP treatment includes a control method comprising the following steps:

determining and/or inputting at least one dosage parameter dependent on patient-related data;

determining and/or inputting at least one application parameter dependent on patient-related data;

providing instructions to administer ICG to the patient; and

providing instructions to apply an output of an application device to a treatment area of the patient; where at least one of the steps is a computer-implemented step.

In some embodiments, a method of treating CNV secondary to AMD includes administering to a patient in need thereof, one or more cytotoxic compounds in combination with i-MP treatment, where the i-MP treatment includes a control method comprising the following steps:

determining and/or inputting at least one dosage parameter dependent on patient-related data;

determining and/or inputting at least one application parameter dependent on patient-related data;

providing instructions to administer ICG to the patient; and

providing instructions to apply an output of an application device to a treatment area of the patient; where at least one of the steps is a computer-implemented step.

In some embodiments, a method of treating CNV secondary to AMD includes administering to a patient in need thereof, one or more immunomodulators in combination with i-MP treatment, where the i-MP treatment includes a control method comprising the following steps:

determining and/or inputting at least one dosage parameter dependent on patient-related data;

determining and/or inputting at least one application parameter dependent on patient-related data;

providing instructions to administer ICG to the patient; and

providing instructions to apply an output of an application device to a treatment area of the patient; where at least one of the steps is a computer-implemented step.

In some embodiments, a method of treating CNV secondary to AMD includes administering to a patient in need thereof, one or more anti-inflammatory compounds in combination with i-MP treatment, where the i-MP treatment includes a control method comprising the following steps:

determining and/or inputting at least one dosage parameter dependent on patient-related data;

determining and/or inputting at least one application parameter dependent on patient-related data;

providing instructions to administer ICG to the patient; and

providing instructions to apply an output of an application device to a treatment area of the patient; where at least one of the steps is a computer-implemented step.

In some embodiments, a method of treating CNV secondary to AMD, includes administration of a medicament, for example an antiangiogenesis compound, in combination with i-MP treatment, where the number of administrations or the dosage of the antiangiogenesis compound required for therapeutic efficacy is less than the number of administrations or the dosage of the antiangiogenesis compound required in the absence of i-MP treatment for the same or similar therapeutic efficacy.

In some embodiments, the invention comprises a method of inhibiting vessel growth and regressing established lesions in AMD, a method of reducing the frequency of dosing of a medicament in treatment of AMD, a method of reducing risk of endophthalmitis and other complications associated with treatment of AMD, a method of improving visual acuity outcomes in treatment of AMD, or a method of reducing patient inconvenience and treatment costs for treatment of AMD comprising

administering one or more medicaments in combination with i-MP treatment, where the i-MP treatment includes a control method comprising the following steps:

determining and/or inputting at least one dosage parameter dependent on patient-related data;

determining and/or inputting at least one application parameter dependent on patient-related data;

providing instructions to administer ICG to the patient; and

providing instructions to apply an output of an application device to a treatment area of the patient; where at least one of the steps is a computer-implemented step, where the reduction or improvement (as appropriate) is from 10-90%.

It is to be understood that the various methods and systems disclosed herein can be used alone, or in any combination, to treat any disease or disorder disclosed herein, or combination thereof, or symptom thereof.

### BRIEF DESCRIPTION OF THE DRAWINGS

Illustrative embodiments of the present invention will be discussed with reference to the accompanying drawings wherein:

FIG. 1A shows schematically a laser system that includes a laser unit and an optical delivery path for delivering laser energy to a patient's eye;

FIG. 1B shows a laser system having a detector positioned at the end of the optical delivery path and providing a feedback signal for calibrating the laser unit;

FIG. 1C is a schematic diagram of an application device including the laser unit of FIG. 1A having a control system, display and user inputs enabling operator interaction with the laser unit;

FIG. 1D is a schematic diagram showing more detail of the system of FIG. 1C;

FIG. 1E is a flowchart diagram of a power control system for use with the laser system;

FIG. 1F is a flowchart showing more detail of the system of FIG. 1E;

FIG. 1G is a graph of the power output of the laser diode in response to a given reference voltage;

FIG. 2 is a flowchart diagram of a mode selection process in the system of FIGS. 1A-1D used as an i-MP application device;

FIG. 3A is a flowchart diagram of steps performed in the AUTO-CALIBRATION mode;

FIG. 3B is a flowchart of a power control system in the AUTO-CALIBRATION mode;

FIG. 3C is a schematic diagram of a generic detector;

FIG. 3D is a schematic diagram of a specific detector;

FIG. 3E is a schematic diagram of the components of a detector;

FIG. 3F is a schematic diagram of a laser calibrator;

FIG. 3G is a block diagram of a laser calibration method;

FIG. 4 is a flowchart diagram of a first set of steps performed in SET Parameter mode;

FIG. 5 is a flowchart diagram of a second set of steps performed in SET Parameter mode;

FIG. 6 is a flowchart diagram of a third set of steps performed in SET Parameter mode;

FIG. 7 is a flowchart diagram of a first set of steps performed in USER Preferences mode;

FIG. 8 is a flowchart diagram of a second set of steps performed in USER Preferences mode;

FIG. 9 is a flowchart diagram of a first set of steps performed in TREATMENT mode;

FIG. 10 is a flowchart diagram of a second set of steps performed in TREATMENT mode;

FIG. 11 is a flowchart diagram of a third set of steps performed in TREATMENT mode;

FIG. 12 is a flowchart diagram of a fourth set of steps performed in TREATMENT mode;

FIG. 13 is a flowchart providing an overview of the therapeutic procedure.

In the following description, like reference characters designate like or corresponding parts throughout the several views of the drawings;

FIG. 14 is a graph illustrating certain results from example 4;

FIGS. 15A and B illustrate the angiography fluorescein of a male patient, of 65 years old, with a visual acuity in right eye of count fingers at 2 mts, presenting a classic CNV. The treatment was i-MP as a monotherapy. FIG. 15A shows the image obtained prior to treatment and FIG. 15B shows the image obtained 10 months after the treatment;

FIGS. 16A and B illustrate the OCT of a male patient, of 65 years old, with a visual acuity in right eye of count fingers at 2 mts, presenting a classic CNV. The treatment was i-MP as a monotherapy. FIG. 16A shows the image obtained prior to treatment and FIG. 16B shows the image obtained 10 months after the treatment;

FIGS. 17A and B show the Angiography fluorescein of a patient, with a visual acuity in the left eye of 0.1, presenting a occult CNV of a patient treated with a combination of i-MP+intravitreal Bevacizumab. FIG. 17A shows the image obtained prior to treatment. FIG. 17B shows the image obtained about 10 months after treatments;

FIGS. 18A and B show the OCT of a patient, with a visual acuity in the left eye of 0.1, presenting a occult CNV of a patient treated with a combined treatment, i-MP+intravitreal Bevacizumab. FIG. 18A shows the image obtained prior to treatment. FIG. 18B shows the image obtained 10 months after treatment; and

FIGS. 19-21 illustrate an exemplary Choroidal Neovascularization and an exemplary variation of the ICG concentration over time in accordance with an exemplary i-MP protocol described herein.

### DETAILED DESCRIPTION OF THE INVENTION

In some embodiments, the invention provides a method of treating AMD, CNV secondary to AMD, exudative AMD (sometimes called neovascular (wet) age-related macular degeneration) including, but not limited to classic and occult CNV, pathological myopia, angioid streaks, syndrome of presumed ocular histoplasmosis, central serous retinopathy, idiopathic polypoidal choroidal vasculopathy, and other conditions resulting from inflammatory and idiopathic causes, that may generate enlargement of abnormal vessels in ocular tissues by administering to a patient in need thereof, at least one medicament, such as, for example, 1-5 medicaments or 1, 2, 3, 4 or 5 medicaments in combination with ICG-mediated photothrombosis (or photothermodynamic) treatment. The i-MP treatment used may vary with certain embodiments (for example, but not limited to, the procedures disclosed in PCT/AU2006/001147, PCT/AU2006/000721 (WO 06/125280), PCT/BR02/00010 (WO 02/094260), Australian Provisional Application No. AU2005904315, Australian Provisional Application No. AU2005902720, Brazilian application No. PI 0102052-8, U.S. Provisional Application No. 60/901,068, other i-MP procedures disclosed herein, or combinations thereof.

In some embodiments, the medicament may be selected from the group consisting of antiangiogenesis compounds, antiproliferative compounds, cytotoxic compounds, immunomodulators, anti-inflammatory compounds and combinations thereof. In some embodiments, the medicament is not an anti-inflammatory or steroid based-compound, for example in some embodiments, the medicament is not triamcinolone or triamcinolone acetonide. In some embodiments, the invention comprises a method of treating CNV secondary to AMD, comprising administering a medicament in combination with i-MP treatment, where the medicament is not an anti-inflammatory compound. Preferably, the medicament is an anti-VEGF angiogenesis inhibitor. Most preferably, the medicament is ranibizumab or bevacizumab. The i-MP treatment used may vary with certain embodiments (for example, but not limited to, the procedures disclosed in PCT/AU2006/001147, PCT/AU2006/000721 (WO 06/125280), PCT/BR02/00010 (WO 02/094260), Australian Provisional Application No. AU2005904315, Australian Provisional Application No. AU2005902720, Brazilian application No. PI 0102052-8, U.S. Provisional Application No. 60/901,068, other i-MP procedures disclosed herein, or combinations thereof.

The medicament may be in any suitable form for administration that achieves the desired result, i.e. inhibition of growth of abnormal vessels and/or destruction of existing abnormal growth. It may be any one or a combination of various therapeutic compounds including small molecule drugs, proteins, peptides, RNA and DNA aptamers, small interfering RNA, antibodies, monoclonal antibodies, antibody fragments, monoclonal antibody fragments, biologically active compounds, ribozymes, plant extracts, vitamins, nutraceuticals, including all analogs of any of the above, pharmaceutically acceptable salts, solvates, hydrates, prodrugs, polymorphs, clathrates, and isotopic variants, including all isomeric forms of the compounds, including optically pure forms, racemates, diastereomers, enantiomers, tautomers and/or mixtures thereof of any of the above. The medicament may be administered through any appropriate route that ensures therapeutic efficacy, such as oral, topical, intravenous, intravitreal, intraocular, sublingual, subcutaneous, intramuscular, intrathecal or any other route of administration. The medicament may be administered according to the standard dosage for the specific route of administration or in any other dose suitable for achieving the desired therapeutic efficacy.

Suitable antiangiogenesis compounds may inhibit formation and growth of blood vessels via any mechanism. For example, suitable anti-VEGF compounds may inhibit the mechanism of action of VEGF either by selectively targeting VEGF-binding sites or by binding to unbound VEGF. Alternatively, anti-VEGF compounds may inhibit the expression of the genes that encode for VEGF or may inhibit any of the steps in the pathway whereby VEGF is produced from the gene or genes, such as by interfering with the mRNA responsible for producing VEGF. Alternatively, such compounds may alter the local environment in such a way as to inhibit blood vessel growth through the promotion of other physical or chemical changes. In addition, such compounds may decreases extracellular protease expression and/or inhibit endothelial cell migration. Non-limiting examples of some suitable antiangiogenesis compounds include: anti-VEGF compounds, such as bevacizumab, ranibizumab, vatalanib and pegaptanib sodium; anti-HIF compounds such as anti-HIF1α compounds, such as topotecan; anti-FGF2 compounds, such as ELR-C—X—C chemokines like Platelet Factor-4 and anti-FGF2 monoclonal antibodies; anti-HGF compounds, such as NK2, NK4 and anti-HGF monoclonal antibodies, squalamine lactate, Candy, EYE001, antiangiogenic steroid-based compounds like anecortave acetate.

Suitable antiproliferative compounds include compounds that inhibit cell growth via any mechanism. For example, suitable antiproliferatives include sirolimus, mycophenolate mofetil and azathioprine.

Suitable cytotoxic compounds include compounds that are toxic to cells, such as abnormally growing cells and include anti-cancer agents such as nitrosourea, cyclophosphamide, melphelan, chlorambucil, platinum compounds, temezolomide and cytotoxic antibiotics such as dactinomycin, anthracyclines, mitomycin C, bleomycin and mithramycin.

Suitable immunomodulators include compounds that alter the functioning of the immune system such as cyclosporine, tacrolimus, daclizumab and pimecrolimus.

Suitable anti-inflammatory compounds include compounds that reduce or prevent inflammation and may include compounds that inhibit tumor necrosis factor or prostaglandin production. Examples of such compounds include infliximib, celecoxib, non-steroidal anti-inflammatory compounds like aceclofenac, acemetacin, acetaminophen, acetaminosalol, acetyl-salicylic acid, acetylsalicylic-2-amino-4-picoline-acid, 5-aminoacetylsalicylic acid, aldlofenac, aminoprofen, amfenac, ampyrone, ampiroxicam, anileridine, bendazac, benoxaprofen, bermoprofen, α-bisabolol, bromfenac, 5-bromosalicylic acid acetate, bromosaligenin, bucloxic acid, butibufen, carprofen, celecoxib, chromoglycate, cinmetacin, clindanac, clopirac, sodium diclofenac, diflunisal, ditazol, droxicam, enfenamic acid, etodolac, etofenamate, felbinac, fenbufen, fenclozic acid, fendosal, fenoprofen, fentiazac, fepradinol, flufenac, flufenamic acid, flunixin, flunoxaprofen, flurbiprofen, glutametacin, glycol salicylate, ibufenac, ibuprofen, ibuproxaam, indomethacin, indoprofen, isofezolac, isoxepac, isoxicam, ketoprofen, ketorolac, lornoxicam, loxoprofen, meclofenamic acid, mefenamic acid, meloxicam, mesalamine, metiazinic acid, mofezolac, montelukast, nabumetone, naproxen, niflumic acid, nimesulide, olsalazine, oxaceprol, oxaprozin, oxyphenbutazone, paracetamol, parsalmide, perisoxal, phenyl-acetyl-salicylate, phenylbutazone, phenylsalicylate, pyrazolac, piroxicam, pirprofen, pranoprofen, protizinic acid, reserveratol, salacetamide, salicylamide, salicylamide-O-acetyl acid, salicylsulphuric acid, salicin, salicylamide, salsalate, sulindac, suprofen, suxibutazone, tamoxifen, tenoxicam, tiaprofenic acid, tiaramide, ticlopridine, tinoridine, tolfenamic acid, tolmetin, tropesin, xenbucin, ximoprofen, zaltoprofen, zomepirac, tomoxiprol, zafirlukast, steroidal anti-inflammatory compounds like cortisol, cortisone, clobetasol, hydrocortisone, fludrocortisone, fludroxycortide, flumetasone, flunisolide, fluocinolone, fluocinonide, fluocortolone, fluorometholone, prednisone, prednisolone, 6-α-methylprednisolone, triamcinolone acetonide, alclometasone, beclometasone, betamethasone, budesonide, dexamethasone, amcinonide, cortivazol, desonide, desoximethasone diflucortolone, difluprednate, fluclorolone and dichlorisone, fluperinidene, fluticasone, halcinonide, meprednisone, methylprednisolone, paramethasone, prednazoline, prednylidene, tixocortol, triamcinolone, and acid derivatives thereof, e.g., acetate, propionate, dipropionate, valerate, phosphate, isonicotinate, metasulfobenzoate, tebutate, and hemisuccinate.

In some embodiments, the method includes treating choroidal neovascularization (CNV), including, but not limited to, classic and occult AMD, that invade the subretinal space or neurosensory tissue resulting from, for example, AMD pathological myopia, angioid streaks, syndrome of presumed ocular histoplasmosis, central serous retinopathy, idiopathic polypoidal choroidal vasculopathy and other conditions from inflammatory and idiopathic causes and other abnormalities that may generate enlargement of abnormal vessels in ocular tissues by administering to a patient in need thereof, at least one medicament in combination with ICG-mediated photothrombosis (i-MP) treatment. In some embodiments, the i-MP treatment is performed on the patient up to 3 weeks before and/or up to 3 weeks after administration of the medicament. In some embodiments, the i-MP treatment comprises a computer-implemented control method. In some embodiments, the i-MP procedures may use computer-implemented control, manual control, or combinations thereof. For example, but not limited to, the procedures disclosed in PCT/AU2006/001147, PCT/AU2006/000721 (WO 06/125280), PCT/BR02/00010 (WO 02/094260), Australian Provisional Application No. AU2005904315, Australian Provisional Application No. AU2005902720, Brazilian application No. PI 0102052-8, U.S. Provisional Application No. 60/901,068, other i-MP procedures disclosed herein. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step.

In some embodiments, a method of treating CNV secondary to AMD comprises administering to a patient in need thereof, one or more antiangiogenesis compounds in combination with i-MP treatment. In some embodiments, the i-MP procedures may use computer-implemented control, manual control, or combinations thereof. The i-MP treatment may include a computer-implemented control method. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step.

In some embodiments, a method of treating CNV secondary to AMD comprises administering to a patient in need thereof, an anti-VEGF compound such as ranibizumab, or bevacizumab, vatalanib or pegaptanib in combination with i-MP treatment. The i-MP treatment may include a computer-implemented control method. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step.

In some embodiments, a method of treating CNV secondary to AMD comprises administering to a patient in need thereof, one or more antiproliferative compounds in combination with i-MP treatment. The i-MP treatment may include a computer-implemented control method. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step.

In some embodiments, a method of treating CNV secondary to AMD comprises administering to a patient in need thereof, one or more cytotoxic compounds in combination with i-MP treatment. The i-MP treatment may include a computer-implemented control method. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step.

In some embodiments, a method of treating CNV secondary to AMD comprises administering to a patient in need thereof, one or more immunomodulators in combination with i-MP treatment. In some embodiments, the i-MP procedures may use computer-implemented control, manual control, or combinations thereof. The i-MP treatment may include a computer-implemented control method. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step.

In some embodiments, a method of treating CNV secondary to AMD comprises administering to a patient in need thereof, one or more anti-inflammatory compounds in combination with i-MP treatment. In some embodiments, the i-MP procedures may use computer-implemented control, manual control, or combinations thereof. The i-MP treatment may include a computer-implemented control method. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step.

In some embodiments, a method of treating CNV secondary to AMD, comprises administration of a medicament to a patient in need thereof, for example an antiangiogenesis compound, in combination with i-MP treatment, where the number of administrations or the dosage of the medicament, such as an antiangiogenesis compound, required for therapeutic efficacy is less than the number of administrations or the dosage of the medicament, such as an antiangiogenesis compound, required in the absence of i-MP treatment for the same or similar therapeutic efficacy, such as no less than 85% of the therapeutic efficacy of the medicament in the absence of i-MP treatment, for example no less than 87%, 90%, 92.5%, 95% or 97.5% of the therapeutic efficacy of the medicament in the absence of i-MP treatment. In some embodiments, the i-MP procedures may use computer-implemented control, manual control, or combinations thereof.

In some embodiments, the invention comprises a method of preventing, inhibiting or reducing vessel growth and regressing established lesions in AMD, the method including administering one or more medicaments in combination with i-MP treatment. The i-MP treatment may include a computer-implemented control method. In some embodiments, the i-MP procedures may use computer-implemented control, manual control, or combinations thereof. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step. The method may reduce vessel growth by 10-100%, such as by 20%, 30%, 33%, 40%, 50%, 60%, 66%, 70%, 75%, 80% or 90% and the size of the established lesions may regress by 10-100%, such as by 20%, 30%, 33%, 40%, 50%, 60%, 66%, 70%, 75%, 80% or 90%.

In some embodiments, the invention comprises a method of reducing the frequency of dosing of a medicament in treatment of AMD when compared to treatment with medicament alone comprising administering one or more medicaments in combination with i-MP treatment. In some embodiments, the i-MP procedures may use computer-implemented control, manual control, or combinations thereof. The i-MP treatment may include a computer-implemented control method. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step. The method may reduce the frequency of dosing of a medicament in treatment of AMD when compared to treatment with medicament alone by 10-100%, such as by 20%, 30%, 33%, 40%, 50%, 60%, 66%, 70%, 75%, 80% or 90%.

In some embodiments, the invention comprises a method of reducing risk of endophthalmitis and other complications associated with treatment of AMD when compared to treatment with medicament alone comprising administering one or more medicaments in combination with i-MP treatment. In some embodiments, the i-MP procedures may use computer-implemented control, manual control, or combinations thereof. The i-MP treatment may include a computer-implemented control method. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step. The method may reduce the risk of endophthalmitis and other complications associated with treatment of AMD when compared to treatment with medicament alone by 10-100%, such as by 20%, 30%, 33%, 40%, 50%, 60%, 66%, 70%, 75%, 80% or 90%.

In some embodiments, the invention comprises a method of reducing patient inconvenience and/or treatment costs for treatment of AMD when compared to treatment with medicament alone comprising administering one or more medicaments in combination with i-MP treatment. In some embodiments, the i-MP procedures may use computer-implemented control, manual control, or combinations thereof. The i-MP treatment may include a computer-implemented control method. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step. The method may reduce patient inconvenience and/or treatment costs when compared to treatment with medicament alone by 10-100%, such as by 20%, 30%, 33%, 40%, 50%, 60%, 66%, 70%, 75%, 80% or 90%.

In some embodiments, the invention comprises a method of improving visual acuity outcomes in treatment of AMD when compared to treatment with medicament alone comprising administering one or more medicaments in combination with i-MP treatment, where the i-MP treatment includes a computer-implemented control method. In some embodiments, the i-MP procedures may use computer-implemented control, manual control, or combinations thereof. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step. The method may improving visual acuity outcomes in treatment of AMD when compared to treatment with medicament alone by 10-100%, such as by 20%, 30%, 33%, 40%, 50%, 60%, 66%, 70%, 75%, 80% or 90%.

In some embodiments, the improvement in visual acuity outcomes associated with the combined i-MP-medicament treatment comprises maintenance of current vision as measured by letters read using the Early Treatment Diabetic Retinopathy Study (ETDRS) visual acuity scale in 75-100% of the treated patients, for example in 75%, 80%, 85%, 90%, 95% or greater of the treated patients. In some embodiments, the improvement in visual acuity outcomes comprises improvement in vision as measured by letters read using the ETDRS visual acuity scale in 40-100% of the treated patients, for example in 40%, 45%, 50%, 55%, 60%, 65%, 70%, 75%, 80%, 85%, 90%, 95% or greater of the treated patients.

In some embodiments, the invention comprises a method of reducing medication error associated with treatment of AMD when compared to treatment with medicament alone comprising administering one or more medicaments in combination with i-MP treatment. In some embodiments, the i-MP procedures may use computer-implemented control, manual control, or combinations thereof. The i-MP treatment may include a computer-implemented control method. In some embodiments, the computer-implemented control method comprises a control method comprising the following steps:

determining and/or inputting at least one dosage parameter, for example 2-10, 3, 4, 5, 6, 7, 8, or 9 dosage parameters, dependent on patient-related data;

determining and/or inputting at least one application parameter, such as 2-10, 3, 4, 5, 6, 7, 8, or 9 application parameters, dependent on patient-related data;

providing instructions to administer ICG to the patient; and

providing instructions to apply an output of an application device to a treatment area of the patient; where at least one of the steps is a computer-implemented step. The method may reduce medication error associated with treatment of AMD when compared to treatment with medicament alone by 10-100%, such as by 20%, 30%, 33%, 40%, 50%, 60%, 66%, 70%, 75%, 80% or 90%.

The medicament may be administered in any suitable form in combination with any suitable excipients one or more times before and/or after i-MP treatment. It may be administered in its standard dose or it may be administered in a reduced dose. In some embodiments, the medicament will be administered intravenously or intravitreally. The combined i-MP-medicament therapy may be administered once every month to once every year, such as, for example, every 2 months, every 3 months, every 4 months, every 5 months, every 6 months, or every 9 months. The combined therapy may include administration of the i-MP treatment up to 1 month before and/or up to 1 month after the medicament treatment.

An example of the combined i-MP-medicament therapy may occur as follows: 1) treatment with i-MP up to 1 month prior to, for example 3 weeks, 2 weeks, 1 week, 6 days, 5 days, 4 days, 3 days, 2 days, 1 day, 12 hours, 6 hours, 4 hours, 3 hours, 2 hours, 1 hour up to immediately before administration of the medicament; 2) administration of the medicament; 3) treatment with i-MP up to 1 month after, for example 3 weeks, 2 weeks, 1 week, 6 days, 5 days, 4 days, 3 days, 2 days, 1 day, 12 hours, 6 hours, 4 hours, 3 hours, 2 hours, 1 hour or immediately after administration of the medicament followed by 4) up to 1 year, for example 9 months, 6 months, 5 months, 18 weeks, 4 months, 14 weeks, 3 months 10 weeks or 2 months before the next administration of the combined i-MP-medicament therapy.

Another example of the combined i-MP-medicament therapy may occur as follows: 1) treatment with i-MP up to 1 month prior to, for example 3 weeks, 2 weeks, 1 week, 6 days, 5 days, 4 days, 3 days, 2 days, 1 day, 12 hours, 6 hours, 4 hours, 3 hours, 2 hours, 1 hour up to immediately before administration of the medicament; 2) administration of the medicament followed by 3) up to 1 year, for example 9 months, 6 months, 5 months, 18 weeks, 4 months, 14 weeks, 3 months 10 weeks, or 2 months before the next administration of the combined i-MP-medicament therapy.

Another example of the combined i-MP-medicament therapy may occur as follows: 1) administration of the medicament; 2) treatment with i-MP up to 1 month, for example 3 weeks, 2 weeks, 1 week, 6 days, 5 days, 4 days, 3 days, 2 days, 1 day, 12 hours, 6 hours, 4 hours, 3 hours, 2 hours, 1 hour or immediately after administration of the medicament followed by 3) up to 1 year, for example 9 months, 6 months, 5 months, 18 weeks, 4 months, 14 weeks, 3 months 10 weeks or 2 months before the next administration of the combined i-MP-medicament therapy.

Accordingly, treatment with the combined i-MP therapy may occur less than 4 times a year, such as less than 3.5, 3.0, 2.5, 2.0, or less than 1.5 times, for example one time a year.

In addition, the frequency of combined i-MP-medicament treatment necessary may reduce with time, for example the treatment frequency necessary in the second year of treatment may be less than the treatment frequency necessary in the first year of treatment and the treatment frequency necessary in the third year may be less than the treatment frequency in the second year. Accordingly, an example of a 4 year treatment protocol may appear as follows: 1 combined treatment every 18 weeks for the first year followed by 1 combined treatment every 24 weeks for the second year, followed by 1 combined treatment every 12 months for the third year, followed by treatment as needed for the fourth and subsequent years.

In one non-limiting example, a method of treating CNV secondary to AMD comprises administering to a patient in need thereof bevacizumab in combination with i-MP treatment. The treatment may occur according to the following regimen: treatment with i-MP, followed by administration of bevacizumab, such as 0.5 to 4.0 mg, for example, 1.0, 1.5, 2.0, 2.5, 3.0 or 3.5 mg, of bevacizumab, within 3 weeks of the i-MP treatment such as within 2 weeks, 1 week, 6 days, 5 days, 4 days, 3 days, 2 days, 1 day, 12 hours, 6 hours, 4 hours, 3 hours, 2 hours, 1 hour of or immediately following the i-MP treatment, followed by repetition of the combined treatment every 12-18 weeks for the first year, every 20-28 weeks, such as every 24 weeks for the second year, every 36 weeks-12 months, such as every 12 months for the third year and as needed for the fourth and subsequent years. The treatment may result in 85%-100%, such as 87.5%, 90%, 92.5%, 95%, or 97.5% of the patients maintaining the same vision as measured by ETDRS letters and 50%-100%, such as 60%, 66%, 70%, 75%, 80%, 85%, 90% or 95% of the patients having improved vision as measured by ETDRS letters.

In another non-limiting example, a method of treating CNV secondary to AMD comprises administering to a patient in need thereof ranibizumab in combination with i-MP treatment. The treatment may occur according to the following regimen: treatment with i-MP, followed by administration of ranibizumab, such as 0.1 to 1.0 mg, for example, 0.2, 0.3, 0.4, 0.5, 0.6, 0.7, 0.8 or 0.9 mg, of ranibizumab, within 3 weeks of the i-MP treatment such as within 2 weeks, 1 week, 6 days, 5 days, 4 days, 3 days, 2 days, 1 day, 12 hours, 6 hours, 4 hours, 3 hours, 2 hours, 1 hour of or immediately following the i-MP treatment, followed by repetition of the combined treatment every 12-18 weeks for the first year, every 20-28 weeks, such as every 24 weeks for the second year, every 36 weeks-12 months, such as every 12 months for the third year and as needed for the fourth and subsequent years. The treatment may result in 85%-100%, such as 87.5%, 90%, 92.5%, 95%, or 97.5% of the patients maintaining the same vision as measured by ETDRS letters and 50%-100%, such as 60%, 66%, 70%, 75%, 80%, 85%, 90% or 95% of the patients having improved vision as measured by ETDRS letters.

In another non-limiting example, a method of treating CNV secondary to AMD comprises administering to a patient in need thereof pegaptanib sodium in combination with i-MP treatment. The treatment may occur according to the following regimen: treatment with i-MP, followed by administration of pegaptanib sodium, such as 0.1 to 1.0 mg, for example, 0.2, 0.3, 0.4, 0.5, 0.6, 0.7, 0.8 or 0.9 mg, of pegaptanib sodium, within 3 weeks of the i-MP treatment such as within 2 weeks, 1 week, 6 days, 5 days, 4 days, 3 days, 2 days, 1 day, 12 hours, 6 hours, 4 hours, 3 hours, 2 hours, 1 hour of or immediately following the i-MP treatment, followed by repetition of the combined treatment every 12-18 weeks for the first year, every 20-28 weeks, such as every 24 weeks for the second year, every 36 weeks-12 months, such as every 12 months for the third year and as needed for the fourth and subsequent years. The treatment may result in 75%-100%, such as 80%, 85%, 87.5%, 90%, 92.5%, 95%, or 97.5% of the patients maintaining the same vision as measured by ETDRS letters and 25%-100%, such as 30%, 33%, 35%, 40%, 50%, 60%, 66%, 70%, 75%, 80%, 85%, 90% or 95% of the patients having improved vision as measured by ETDRS letters.

In yet another non-limiting example, a method of treating CNV secondary to AND comprises administering to a patient in need thereof anecortave acetate in combination with i-MP treatment. The treatment may occur according to the following regimen: treatment with i-MP, followed by administration of anecortave acetate, such as 0.1 to 1.0 mg, for example, 0.2, 0.3, 0.4, 0.5, 0.6, 0.7, 0.8 or 0.9 mg, of anecortave acetate, within 3 weeks of the i-MP treatment such as within 2 weeks, 1 week, 6 days, 5 days, 4 days, 3 days, 2 days, 1 day, 12 hours, 6 hours, 4 hours, 3 hours, 2 hours, 1 hour of or immediately following the i-MP treatment, followed by repetition of the combined treatment every 12-18 weeks for the first year, every 20-28 weeks, such as every 24 weeks for the second year, every 36 weeks-12 months, such as every 12 months for the third year and as needed for the fourth and subsequent years. The treatment may result in 75%-100%, such as 80%, 85%, 87.5%, 90%, 92.5%, 95%, or 97.5% of the patients maintaining the same vision as measured by ETDRS letters and 25%-100%, such as 30%, 33%, 35%, 40%, 50%, 60%, 66%, 70%, 75%, 80%, 85%, 90% or 95% of the patients having improved vision as measured by ETDRS letters.

In another non-limiting example, a method of treating CNV secondary to AMD comprises administering to a patient in need thereof triamcinolone or triamcinolone acetonide in combination with i-MP treatment. The treatment may occur according to the following regimen: treatment with i-MP, followed by administration of triamcinolone or triamcinolone acetonide, such as 2.0-10.0 mg, for example, 3.0, 3.5, 4.0, 4.5, 5.0, 5.5, 6.0, 6.5, 7.0, 7.5, 8.0, 8.5, 9.0 or 9.5 mg, of triamcinolone or triamcinolone acetonide, within 3 weeks of the i-MP treatment such as within 2 weeks, 1 week, 6 days, 5 days, 4 days, 3 days, 2 days, 1 day, 12 hours, 6 hours, 4 hours, 3 hours, 2 hours, 1 hour of or immediately following the i-MP treatment, followed by repetition of the combined treatment every 12-18 weeks for the first year, every 20-28 weeks, such as every 24 weeks for the second year, every 36 weeks-12 months, such as every 12 months for the third year and as needed for the fourth and subsequent years. The treatment may result in 85%-100%, such as 87.5%, 90%, 92.5%, 95%, or 97.5% of the patients maintaining the same vision as measured by ETDRS letters and 50%-100%, such as 60%, 66%, 70%, 75%, 80%, 85%, 90% or 95% of the patients having improved vision as measured by ETDRS letters

In another non-limiting example, a method of treating CNV secondary to AMD comprises administering to a patient in need thereof vatalanib in combination with i-MP treatment. The treatment may occur according to the following regimen: treatment with i-MP, followed by administration of vatalanib, such as 0.1 to 2.0 g, for example 0.2, 0.3, 0.4, 0.5, 0.6, 0.7, 0.8, 0.9, 1.0, 1.25, 1.50 or 1.75 g, of vatalanib, within 3 weeks of the i-MP treatment such as within 2 weeks, 1 week, 6 days, 5 days, 4 days, 3 days, 2 days, 1 day, 12 hours, 6 hours, 4 hours, 3 hours, 2 hours, 1 hour of or immediately following the i-MP treatment or within 1, 2 or 3 weeks of the treatment, followed by repetition of the combined treatment every 12-18 weeks for the first year, every 20-28 weeks, such as every 24 weeks for the second year, every 36 weeks-12 months, such as every 12 months for the third year and as needed for the fourth and subsequent years. The treatment may result in 85%-100%, such as 87.5%, 90%, 92.5%, 95%, or 97.5% of the patients maintaining the same vision as measured by ETDRS letters and 50%-100%, such as 60%, 66%, 70%, 75%, 80%, 85%, 90% or 95% of the patients having improved vision as measured by ETDRS letters.

In some embodiments, the medicament may additionally be administered one or more times between administrations of the combined i-MP-medicament therapy and may be, administered, for example, every month, every 2 months, every 10 weeks, every 3 months, every 14 weeks, every 4 months, every 18 weeks, every 5 months, every 6 months, or every 9 months after the combined i-MP-medicament therapy. In some embodiments, i-MP treatment may additionally be administered one or more times between administrations of the combined i-MP-medicament therapy and may be administered, for example, every month, every 2 months, every 10 weeks, every 3 months, every 14 weeks, every 4 months, every 18 weeks, every 5 months, every 6 months, or every 9 months after the combined i-MP-medicament therapy.

In some embodiments, the medicament therapy may be on one cycle of treatment such as once every month, every six weeks, every two months, every ten weeks, every 3 months, every 14 weeks every 4 months, every 18 weeks, every 5 months every 6 months, every 9 months or every year, while the i-MP therapy is independently on a different cycle of treatment beginning before or after the medicament therapy and may independently be administered once every month, every 6 weeks, every two months, every ten weeks, every 3 months, every 14 weeks, every 4 months, every 18 weeks, every 5 months every 6 months, every 9 months or every year.

In some embodiments, the combination of administration of the medicament with i-MP treatment may reduce the number of administrations or dosage of treatment necessary to achieve therapeutic efficacy when compared to the number of administrations or dosage of treatment in the absence of either i-MP or medicament for the same therapeutic efficacy.

In some embodiments of the methods of treatment, the dosage of the medicament required for the same therapeutic efficacy as the medicament alone may be reduced by 10 to 75%, for example reduced by 20%, 30%, 33%, 40%, 50% 60%, 66% or 70%. In some embodiments of the methods of treatment, the frequency of administration of the medicament required for the same therapeutic efficacy as the medicament alone may be reduced by 10 to 75%, for example reduced by 20%, 30%, 33%, 40%, 50% 60%, 66% or 70%. In some embodiments the frequency of the i-MP treatment required for the same therapeutic efficacy as i-MP alone may be reduced by 10 to 75%, for example reduced by 20%, 30%, 33%, 40%, 50% 60%, 66% or 70%.

By reducing the dosage or the frequency of administration, the methods of the invention reduce the occurrence of any one or any combination of the following potential adverse events increased risk of stroke, myocardial infarction, vascular death, arterial thromboembolic events, endophthalimitis, conjunctival hemorrhage, eye pain, vitreous floaters, retinal hemorrhage, increase in intraocular pressure, vitreous detachment, intraocular inflammation, eye irritation, cataract, foreign body sensation in eyes, increase in lacrimation, eye pruritis, visual disturbance, blepharitis, subretinal fibrosis, ocular hyperemia, maculopathy, blurring of or decrease in visual acuity, detachment of the retinal pigment epithelium, dry eye, ocular discomfort, conjunctival hyperemia, posterior capsule opacification, retinal exudates, hypertension/elevated blood pressure, nasopharyngitis, arthralgia, headache, bronchitis, cough, anemia, nausea, sinusitis, upper respiratory tract infection, back pain, urinary tract infection, influenza, arthritis, dizziness, and constipation by 10-100%, for example reduced by 20%, 30%, 33%, 40%, 50% 60%, 66%, 70%, 75%, 80% or 90%.

Treatment Using i-MP

In some embodiments, the invention provides a method of treating CNV secondary to AMD and associated disorders using a combination of a therapeutic agent and an i-MP system. The i-MP laser system according to some aspects of the invention may be used in conjunction with ICG for photodynamic treatment of vascular occlusion of choroidal vessels or neovascularization originating from choroids that invade the subretinal tissue or neurosensory tissue itself, in a selective form, achieving intravascular coagulation by a photothermodynamic effect without significantly damaging, heating and/or hyperthermy of adjacent tissues. In some embodiments the i-MP laser system may be used in the treatment CNV secondary to wet AMD (e.g., wet AMD with classic subretinal CNV (“wet classic AMD”), wet AMD with occult subretinal CNV (“occult CNV”), or wet AMD with classic and occult subretinal CNV (“mixed CNV”) (including, but not limited to, predominantly classic or minimally CNV lesions) in a patient (e.g., a human), using the system and/or computer program product described herein.

Therefore, the i-MP laser treatments and system for use in some embodiments of the present invention have at least one or more of the following purposes:
1) Treatment of abnormal vessels of ocular tissues, particularly in choroids, retina and/or between the choroids and the retina, which are leaking fluid and/or blood to the referred tissues, and thereby, increasing the risk and/or causing visual decay.
2) Use of an infrared laser in wavelength band that has a selective affinity or absorption with a photosensitizing dye such as ICG.
3) Use of an ICG dye in its natural form and/or modified by liposome encapsulation and other chemical alterations, such as lyophylization, thereby inducing diminishment of aggregation or intravascular selectivity and a high photophysical and photochemical reaction affinity.
4) Use of delivery systems for light excitation for the treatment methods herein that minimize or eliminate thermal tissue reaction, typical of photocoagulation, causing only, photodynamic effect and/or hyperthermy that generates temperature increases insufficient for clotting or burning adjacent tissues.
5) Treatment of AMD and related ocular disorders using an ambulatory and/or surgical treatment process that does not require anesthesia, using a projection of laser light that does not have a photothermodynamic effect until the light excitation activates a photosensitizing agent. Activation of the photosensitizing agent, such as ICG occurs in vivo, after light exposure at a dose, intensity, luminance and power, in accordance with the best selective occlusion effect.
6) Use of a contact or non-contact lens for focalization of the source of infrared laser on the targeted tissue to be treated.
7) Administration of a photosensitizing substance with light excitation for a desired therapeutic effect.
8) Use of a system of specific focalization of laser light for larger concentration of a photosensitive dye on the targeted tissue, for achieving the tissue reaction induced by photodynamic reaction.

The procedures for treatment using i-MP may include some or all of the following steps:
1) administration of a photosensitizing agent;
2) waiting until photosensitizing agent has reached a proper maximum distribution and localization inside a pre-selected area for treatment;
3) optionally repeating the injection according to the case and/or cause of the vascular alteration to be treated.
4) using a medical instrument that includes an infrared laser light source, to induce an interaction between the light and the photosensitizing agent, for a specific length of time.
5) excitation of the photosensitizing agent concentrated in the targeted tissue, during the predetermined time while observing the tissues being treated and any visible direct or indirect effect by means of an optical instrument coupled to the emitting source of laser light.

Some embodiments of the present invention, therefore, include the use of an energy source such as an infrared wavelength laser to activate a photosensitizing agent such as ICG that has been administered intravenously to a patient by absorption of the laser light by the agent. This absorption occurs under controlled conditions that avoid photocoagulation while promoting, via photophysical and photochemical interactions, a photodynamic effect that results in insufficient hyperthermy to cause damage to tissue adjacent to the tissue that is treated while achieving the desired therapeutic effect, such as treatment of CNV secondary to AMD.

In some embodiments, the decrease of the thermal component in the mechanism of action may be selectively controlled to produce a smaller photodynamic effect, while still using the same laser/photosensitizing agent combination.

In some embodiments, the i-MP procedures may use computer-implemented control, manual control, or combinations thereof. For example, but not limited to, the procedures disclosed in PCT/AU2006/001147, PCT/AU2006/000721 (WO 06/125280), PCT/BR02/00010 (WO 02/094260), Australian Provisional Application No. AU2005904315, Australian Provisional Application No. AU2005902720, Brazilian application No. PI 0102052-8, U.S. Provisional Application No. 60/901,068, and other i-MP procedures disclosed herein. An example of an i-MP procedure that may be used is attached and entitled “Protocol for the Evaluation of the Indocyanine Green-mediated Photothrombosis (i-MP), in the Treatment of the Occult Subfoveal Neovascular Membranes of the Age-Related Macular Degeneration (AMD)”, this procedure may be used in combination with other certain embodiments disclosed.

### Characteristics of the Laser

In some embodiments, the energy source (or activation source) is a diode laser having a wavelength of from 700 to 900 nm, for example from 750 to 850 nm, such as 800 nm to 825 nm, 805 to 815 nm or 810 nm. Preferably, the wavelength of the laser is 805 nm or 810 nm, 805 nm being the maximum absorption peak for ICG. In order to minimize photocoagulation and collateral tissue damage and to promote photodynamic effects, the laser should be used at powers of less than 1.0 W, such as less than 900 mW, less than 800 mW, less than 750 mW, less than 600 mW, less than 500 mW, less than 400 mW, less than 300 mW, less than 200 mW or less than 100 mW and the exposure time is from about 30 seconds to about 180 seconds, for example 40, 50, 60, 70, 80, 90, 100, 120, 130, 140, 150, 160, 170 or 180 seconds. The laser may treat areas of circular application no less than 1.6 mm in diameter, for example 1.6 to 8.6 mm, such as greater than 1.6 mm, greater than 2.0 mm, greater than 2.5 mm, greater than 3.0 mm, greater than 4.0 mm, greater than 5.0 mm, greater than 6.0 mm, greater than 7.0 mm, greater than 8.0 mm, or 8.6 mm in diameter, resulting in retinal irradiances that are never greater than 4.0 W/cm². The beam may be round or modified and may be customized in accordance with the targeted tissue. In some embodiments the diode laser may have a diaphragm mechanism with different openings for exposure of beams with different diameters such as 0.8, 1.0, 1.2, 1.5, 2.5 and 4.3 mm, coupled to a co-observation system with a biomicroscope in slit-lamp form.

By using such a laser under such conditions, the activation of the ICG may be controlled such that the response is primarily by means of photo-oxidation effects type II, resulting in acuity stabilization or improvement; in general, with improvement of relative scotome, with no collateral damages and with low rates of retreatment of the subretinal neovascular membrane required.

In some embodiments, successful treatment may be achieved with irradiation as low as 7.2 Joules/cm².

### Indocyanine Green

ICG constitutes a relatively cheap and safe medicament having a very low local and systemic toxicity and a favorable biodistribution, as well as known fluorescence properties suitable for use with improved digital detection technologies and video angiography cameras able to actuate in the infrared wavelength band. Water-solubility, fluorescent properties and activation at 805 nm make ICG an ideal photosensitizer for choroidal neovascularization. In particular, its fluorescence properties assure the exact contrast localization in subretinal neovascular lesions, prior to positioning the laser beam for application of activating light.

ICG is commercially available, and distributed in a generic and ample form, in all opthalmologic markets. The medication is easily administered, and is rapidly distributed and excreted. Up to now, ICG was used for photodynamic therapy treatment in combination with antiangiogenesis drug therapies or using an automatic laser control and calibration system. ICG may be provided as a powder in a vial, such as a freeze dried product in amounts of 10-150 mg, for example as 10, 15, 25, 50, 75, 100 or 150 mg of ICG/vial. The dosages used may vary from patient to patient based on a variety of parameters. For example, for patients less than 75 kg in weight, 100 mg of ICG may be used, while for patients greater than 75 kg in weight, 150 mg of ICG may be used. Distilled water may be added to the vials immediately prior to use to reconstitute the ICG and prepare it for use.

ICG is a water-soluble tricarbocyanine molecule that does not contain more than 5% sodium chloride, and is chemically named 4-(2-{7-[1,1-Dimethyl-3-(4-sulfo-butyl)-1H-benzo[e]indol-2-yl]-hepta-2,4,6-trienylidene}-1,1-dimethyl-benzo[e]indolium-3-yl)-butane-1-sulfonic acid sodium salt. It has the following empirical formula: C₄₃H₄₇N₂NaO₆S₂, giving it a molecular weight of 774.98.

In addition to having a high molecular weight, ICG is bound in vivo rapidly and almost completely (98%) to plasma proteins. Albumin is its major carrier; however, in human serum, 80% of ICG can be bound to globulins, probably alpha-1 lipoproteins (Baker K J., Binding of sulphobromophtalein (BSP) sodium and ICG (ICG) by plasma alpha-1 lipoproteins, Proc Soc Exp Biol Med 1966, 122:957-963) that have hindered vascular retention and minimal leakage from abnormal vessels (Ham W, Sliney D., Retinal sensitivity to damage from short wavelength light, Nature 1976, 260:153-155). Like choroidal neovascularization, tumor neovasculature is highly proliferative. (Denekamp J., Vascular attack as a therapeutic strategy for cancer, Cancer Metastasis Rev 1990, 9:267-282). The hyperproliferative neovascular tissue exhibits an enhanced permeability and elevated levels of specific albumin and LDL receptors (Schmidt U, Birngruber R, Hasan T, Selective occlusion of ocular neovascularization by photodynamic therapy, Opthalmology 1992, 89: 391-394; Rutledge J, Curry F, Blanche P, Krauss R., Solvent drag of LDL across mammalian endothelial barriers with increased permeability, Am J Physiol 1995, 268:H1982-H1991) leading to increased LDL transport across endothelial junctions (Schnitzer J, Carley W, Palade G., Albumin interacts with a 60-kDa microvascular endothelial glycoprotein, Proc. Natl. Acad. Sci. 1988; 85:6773-6777).

### Mechanism of Action of ICG

While not being bound by any theory, it is believed that ICG may operate via two mechanisms. These mechanisms have been identified as photooxidation reaction types I and II.

In photooxidation reaction type I, ICG is believed to release its absorbed energy via a non-radioactive mechanism, where the molecule releases the energy absorbed in the form of heat, by internal conversion or by transfer to other molecules, thereby damaging cells by raising their intracellular temperature, resulting in photocoagulation (Reichel E, Puliafito C, Duker J, Guyer D., ICG dye enhanced diode photocoagulation of poorly defined subfoveal choroidal neovascularization, Ophthalmic Surg., 1994, 25: 195-201) or tissue welding (Decoste S, Farinelli W, Flotte T, Anderson R., Dye-enhanced laser welding for skin closure, Laser Surg Med 1992, 12: 25-32). Generally, this mechanism results upon absorption of higher amounts of energy than those used in the current invention and causes damage to adjacent tissues.

Alternatively, ICG dye may release the energy absorbed by transfer to an oxygen molecule by means of a photo-oxidation type II via a triplet state, and to other components to form reactive intermediates such as singlet oxygen, which can cause irreversible destruction in biological substrates. Other reactive species such as superoxide, hydroperoxyl, or hydroxyl radicals may also be involved in the irreversible damage mediated. (Henderson B W, Dougherty T J., How does photodynamic therapy work?, Photochem Photobiol 1992, 55: 145-157, Roberts W, Hasan T., Role of neovasculature and vascular permeability on the tumor retention of photodynamic agents, Cancer Res 1992, 52: 924-930). Because singlet oxygen flows at a reactive distance of only 0.1 μm, the cytotoxicity is restricted to the immediate vicinity of the photoactivated photosensitizer. At the doses of photosensitizer and light used herein, neither the light nor the sensitizer has any independent activity contrary to the targeted tissue. Occlusion of the vascular bed reached as a major mechanism of action of the photodynamic therapeutic process, arises after damage to endothelial cells with subsequent platelet adhesion and thrombus formation. Indirect evidences suggest that the primary photodynamic reaction is a type II photosensitization mediated by singlet oxygen.

Studies with photosensitizers have shown differences in singlet oxygen quantum: 0.29 for a hematoporphyrin derivative; 0.36 for phthalocyanines; 0.67 for purpurins; 0.77 for NPe₆. The triplet oxygen quantum by ICG is 0.14 in water and 0.16 in methanol, and 0.17 in dimethyl sulphoxide (Reindl S, Penzkofer Ar Gong S—H, et al., Quantum yield of triplet formation for ICG, J Photochem Photobiol A 1997, 105: 65-68).

In comparison to other photosensitizers, the triplet energetic fields of ICG appear low, but unexpectedly it has been found that they are sufficiently high to induce photodynamic therapy in AMD and related disorders.

ICG is not metabolized after an intravenous injection, and is excreted exclusively through hepatic means. It is not absorbed from the intestine and does not stay in enterohepatic circulation. The dye is taken from the plasma by the hepatic parenchyma cells and secreted into the bile. Negligible uptake of the dye occurs in the kidneys, lungs, cerebrospinal fluid, and peripheral circulation. Renal excretion does not occur. The safety of intravenous administration of ICG in humans is well documented, with severe adverse reactions occurring in only 0.05% of patients. Although greater concentration levels of the second-generation agents for photodynamic therapy with benzoporphyrins, purpurins, phthalocyanines, mono-L-aspartyl-chlorine-6 were determined in experimental studies, the distribution and retention of these drugs in human neovascular tissue is currently extrapolated from pretreatment conditions, reached by means of fluorescein angiography and the concentration of sodium fluorescein in neovascular membrane rather than concentration of the photosensitive substance in neovascular membrane.

These data may not properly reflect the biodistribution of the photosensitizer in the eye and its association with the targeted field to be treated. This lack is not an issue with ICG because of the existence of an angiographic method for investigation of its concentration prior to the treatment itself, and thus, the uptake specificity of the neovascular membrane for this photosensitive dye can be determined, prior to treatment. Taking a particular patient as basis, it is possible to associate the properties of fundus imaging concomitant with the phototherapeutic properties of ICG to optimize either the propaedeutic or disease treatment in only one step. Photodynamic therapy with other ophthalmic agents is mainly limited by poor aqueous solubility, needing intralipid or liposomal formulation and consequent intravenous administration by slow infusion. In contrast, water soluble ICG can be rapidly infused, thereby avoiding long intervals of 10 minutes or more, required for slow infusion of a preparation for suspension liposomes and/or with intralipid formulation. This property also enhances treatment practicality and promotes, improves and increases patient compliance and comfort. In addition, modifications of ICG with encapsulation and other chemical modification can also be rapidly administered.

ICG reveals a high absorption and therefore high activation in the infrared spectrum region around 805 nm, making it an attractive target for commercial 810 nm lasers. Infrared light penetrates deeper into tissue of red light, thereby conferring advantages in the selectivity for treatment of subretinal or choroidal neovascular membranes. Furthermore, its spectral penetration characteristics facilitate the use of angiography with prior delineation of the choroidal tissue and/or choriocapillaris and/or neovascularization for the subsequent treatment.

The ICG may be administered intravenously at doses from 0.5 to 5 mg/kg such as such as 1 mg/kg, 2 mg/kg, 3 mg/kg or 4 mg/kg. In certain embodiments, the total ICG dose administered may be about 40 mg, about 50 mg, about 60 mg, about 75 mg, about 85 mg, about 100 mg, about 125 mg, about 150 mg or about 200 mg. In certain embodiments, the doses may be prepared in any suitable packaging and may be administered immediately before application, up to 30 minutes before application of the laser or both 30 minutes before application of the laser and immediately before application of the laser.

Controlling the i-MP System

The controlling of the i-MP system may vary in certain embodiments. In certain embodiments the control system may be characterized as a manual control system, a substantially manual control system, a partially computer implemented control system, a substantially computer implemented control system, or combinations thereof. For example, but not limited to, the procedures disclosed in PCT/AU2006/001147, PCT/AU2006/000721 (WO 06/125280), PCT/BR02/00010 (WO 02/094260), Australian Provisional Application No. AU2005904315, Australian Provisional Application No. AU2005902720, Brazilian application No. PI 0102052-8, U.S. Provisional Application No. 60/901,068 and other i-MP procedures disclosed herein.

In some embodiments, the i-MP system may be controlled using a computer-implemented method of controlling a therapeutic procedure performed on a patient, where the method comprises:

determining at least one dosage parameter and at least one application parameter of the therapeutic procedure dependent on patient-related data;

displaying one or more prompts instructing an operator to introduce ICG into the patient in accordance with the at least one dosage parameter; and

presenting one or more instructions to the operator to apply an output of an application device to a treatment area of the patient in accordance with the at least one application parameter.

According to a further aspect of the i-MP system there is provided a computer-implemented method of controlling a therapeutic procedure performed on a patient, the method comprising:

determining, dependent on patient-related data, a dosage of an external substance, such as a photosensitizing agent to be introduced into the patient;

calculating, dependent on the patient-related data, a desired output of an application device, such as an i-MP laser treatment device, to be applied to a treatment area of the patient;

displaying prompts instructing an operator to introduce the external substance into the patient in accordance with a timing schedule of the therapeutic procedure; and

presenting instructions to the operator to apply the output of the application device to the treatment area, the instructions being presented according to the timing schedule.

In other aspects of the i-MP system, there is provided a computer-implemented method of controlling a procedure for treating macular degeneration in a patient's eye, the method comprising:

receiving data relating to the patient;

determining a quantity of an external substance, such as ICG, to be introduced into the patient dependent on the received data;

calculating a desired power output of a laser to be applied to a treatment area in the patient's eye;

displaying prompts instructing an operator to introduce the external substance into the patient in a plurality of doses, wherein the prompts are displayed according to a timing schedule; and

presenting instructions to the operator to apply the laser beam to the treatment area in a plurality of applications, the instructions being presented according to the timing schedule.

In a further aspect, there is provided a system for controlling a therapeutic procedure performed on a patient, the system comprising:

means for determining at least one dosage parameter and at least one application parameter of the therapeutic procedure, such as i-MP treatment, dependent on patient-related data;

means for displaying one or more prompts instructing an operator to introduce at least one external substance, such as ICG, into the patient in accordance with the at least one dosage parameter; and

means for presenting one or more instructions to the operator to apply an output of an application device to a treatment area of the patient in accordance with the at least one application parameter.

In another aspect, there is provided a system for controlling a therapeutic procedure, such as i-MP treatment, performed on a patient, the system comprising:

data storage for storing patient-related information;

a display for displaying information to an operator; and

a processor in communication with the data storage and the display and arranged to:

determine at least one dosage parameter and at least one application parameter of the therapeutic procedure dependent on the patient-related data;

cause the display of one or more prompts instructing an operator to introduce at least one external substance into the patient in accordance with the at least one dosage parameter; and

cause the display of one or more instructions to the operator to apply an output of an application device to a treatment area of the patient in accordance with the at least one application parameter.

According to a further aspect of the i-MP system there is provided a computer program product comprising machine-readable program code recorded on a machine-readable recording medium, for controlling the operation of a data processing apparatus on which the program code executes to perform a method of controlling a therapeutic procedure, such as i-MP treatment, performed on a patient, the method comprising:

determining at least one dosage parameter and at least one application parameter of the therapeutic procedure dependent on patient-related data;

displaying one or more prompts instructing an operator to introduce at least one external substance, such as ICG, into the patient in accordance with the at least one dosage parameter; and

presenting one or more instructions to the operator to apply an output of an application device to a treatment area of the patient in accordance with the at least one application parameter.

According to a further aspect of the i-MP system there is provided a computer program product comprising machine-readable program code recorded on a machine-readable recording medium, for controlling the operation of a data processing apparatus on which the program code executes to perform a method of controlling a therapeutic procedure, such as i-MP treatment, performed on a patient, the method comprising:

determining, dependent on patient-related data, a dosage of an external substance, such as ICG, to be introduced into the patient;

calculating, dependent on the patient-related data, a desired output of an application device to be applied to a treatment area of the patient;

displaying prompts instructing an operator to introduce the external substance into the patient in accordance with a timing schedule of the therapeutic procedure; and

presenting instructions to the operator to apply the output of the application device to the treatment area, the instructions being presented according to the timing schedule.

According to a further aspect of the i-MP system there is provided a computer program comprising machine-readable program code for controlling the operation of a data processing apparatus on which the program code executes to perform a method of controlling a therapeutic procedure, such as i-MP treatment, performed on a patient, the method comprising:

determining at least one dosage parameter and at least one application parameter of the therapeutic procedure dependent on patient-related data;

displaying one or more prompts instructing an operator to introduce at least one external substance, such as ICG, into the patient in accordance with the at least one dosage parameter; and

presenting one or more instructions to the operator to apply an output of an application device to a treatment area of the patient in accordance with the at least one application parameter.

According to another aspect, there is provided a method of controlling a therapeutic procedure performed on a patient, such as i-MP treatment, comprising determining and/or inputting at least one dosage parameter dependent on patient-related data; determining and/or inputting at least one application parameter dependent on patient-related data; providing instructions to administer at least one external substance, such as ICG into the patient; and providing instructions to apply an output of an application device to a treatment area of the patient, where at least one of the steps is a computer-implemented step.

According to another aspect, there is provided a computer program product comprising machine readable program code recorded on a machine readable recording medium, for controlling the operation of a data processing apparatus on which the program code executes to perform a method of controlling a therapeutic procedure performed on a patient, such as i-MP treatment, comprising determining and/or inputting at least one dosage parameter dependent on patient-related data; determining and/or inputting at least one application parameter dependent on patient-related data; providing instructions to administer at least one external substance, such as ICG into the patient; and providing instructions to apply an output of an application device to a treatment area of the patient, where at least one of the steps is a computer-implemented step.

In embodiments, all of the steps may be computer-implemented steps or at least one of the steps may be a manually implemented step. The method may be used to treat at least one of the following: CNV secondary to wet AMD, Angioid Streaks, Pathologic Myopia, Central Serous Retinopathy, or other choroidal diseases resulting from inflammatory conditions and idiopathic causes as compared to conventional systems. In certain embodiments, wet AMD may include any one of classic CNV, occult CNV, mixed forms of CNV, predominantly classic CNV, predominantly occult CNV, Polypoidal CNV and combinations of any of the above.

In certain embodiments, the therapeutic procedure may have a timing sequence and providing steps may display prompts and present instructions according to the timing sequence. In certain embodiments, the method may also include interrupting the therapeutic procedure if at least one specified action is not completed within a specified time and/or requesting the operator to enter the patient-related data. In certain embodiments, the method may also include adjusting at least one setting of the application device dependent on the at least one application parameter, and the output of the application device may be dependent on the at least one setting.

In certain embodiments, the application device may be a laser and the adjusting step may adjust a power output of the laser.

In certain embodiments, the method may also include providing instructions for the operator to operate the application device according to a predetermined calibration procedure to calibrate the application device for the therapeutic procedure.

In certain embodiments, the method may also include displaying safety information related to the therapeutic procedure, the safety information being displayed at one or more predetermined stages of the therapeutic procedure.

In certain embodiments, the application device may comprise a laser.

In certain embodiments, the patient-related data may include at least one of: a weight of the patient; a maximum dimension of a lesion in an eye of the patient; and a level of pigmentation in the eye of the patient.

In certain embodiments, the dosage parameter may be a quantity of the external substance to be administered into the patient.

According to another aspect of the invention there is provided a method of reducing medication error including utilizing a computer to control at least a portion of a therapeutic procedure.

In certain embodiments, a dosage of ICG may be administered from a single vial to reduce medication error associated with i-MP treatment.

In certain embodiments, the computer controlled portion of the therapeutic procedure may be more precise and require less operator intervention and/or discretion as compared with conventional systems of treatment using i-MP.

In certain embodiments, the medication error may be reduced by any of the following amounts: between about 5% to about 25%; between about 10% to about 35%; between about 25% to about 60%; or between about 25% to about 90% as compared with conventional methods of treatment using i-MP.

In certain embodiments, the medication error may be reduced by any of the following amounts: greater than about 5%; greater than about 15%; greater than about 25%; greater than about 35%; greater than about 60%; greater than about 80%; or greater than about 95% as compared with conventional methods of treatment using i-MP or other.

Accordingly, combined computer-implemented i-MP-medicament treatment may synergistically provide reduction of medication error from 10% to about 100%, such as 15%, 20%, 25%, 30%, 33%, 40%, 50%, 60%, 67%, 70%, 75%, 80%, 85%, 90%, 95%, 97.5% or 99% reduction in medication error when compared to non-computer-implemented i-MP treatment alone and medicament treatment alone.

According to another aspect, there is provided a system and method, such as the control system for i-MP treatment, that provides one or more of the following: additional safety, reduction in medication error, higher efficacy, fewer side effects, less damage to healthy retinal tissue and suitable treatment for all kinds of CNV secondary to AMID, Angioid Streaks, Pathologic Myopia, Central Serous Retinopathy, and other choroidal diseases resulting from inflammatory conditions and idiopathic causes as compared to conventional systems.

According to another aspect, the automation introduced by the methods and systems disclosed allow for a broader range of physicians/operators with varying levels of skill and experience to treat patients and these treatments may generally be more effective as a whole.

According to another aspect, the methods and systems disclosed allows doctors or operators to perform the disclosed treatments without having to use specialized diagnostic equipment such as ICG angiography imaging systems and similar equipment.

The systems and methods disclosed herein may be used in the application of any suitable therapeutic procedures, such as those described in WO 2006/125280 and in WO 02/094260. Specifically, for example, the systems and methods disclosed herein can be used to treat CNV secondary to wet AMD, including but not limited to, classic CNV, wet occult CNV, mixed forms of CNV, predominantly or minimally classic CNV lesions, and/or combination of any of the above.

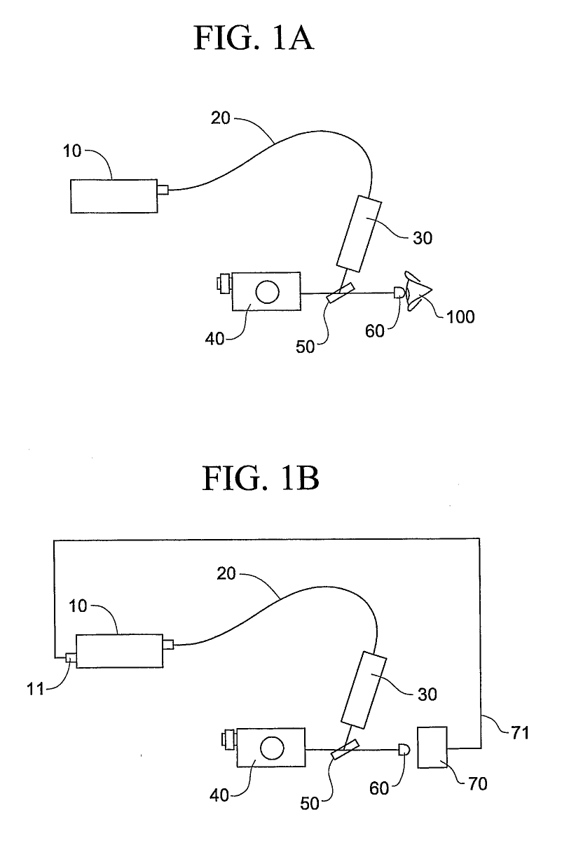

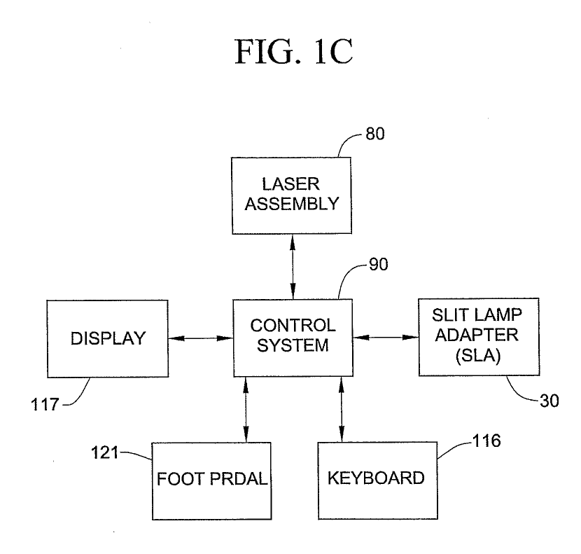

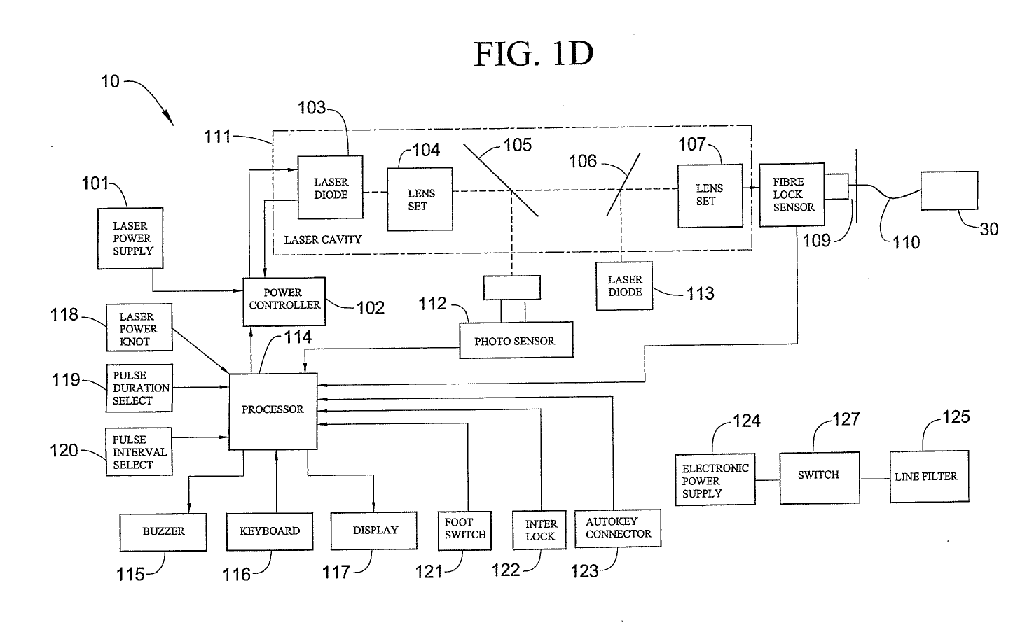

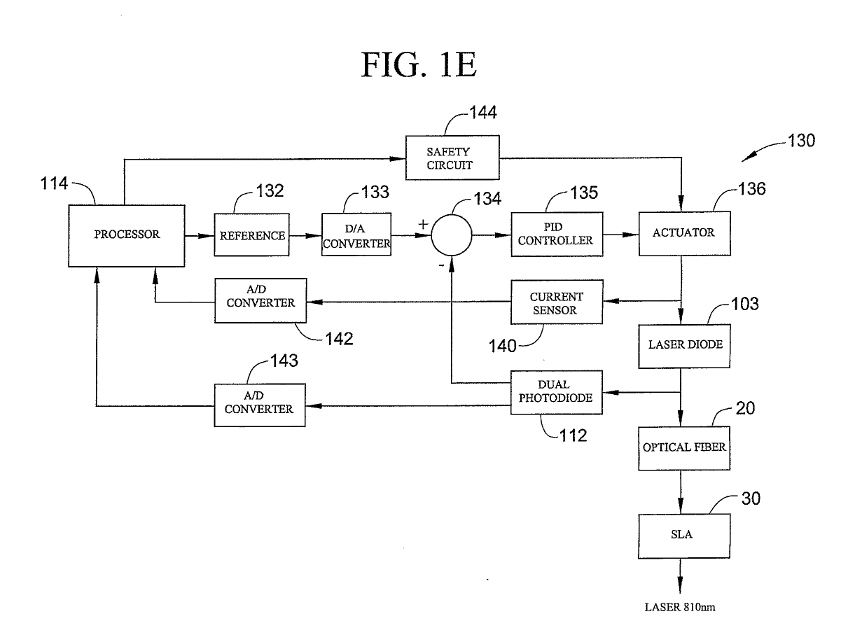

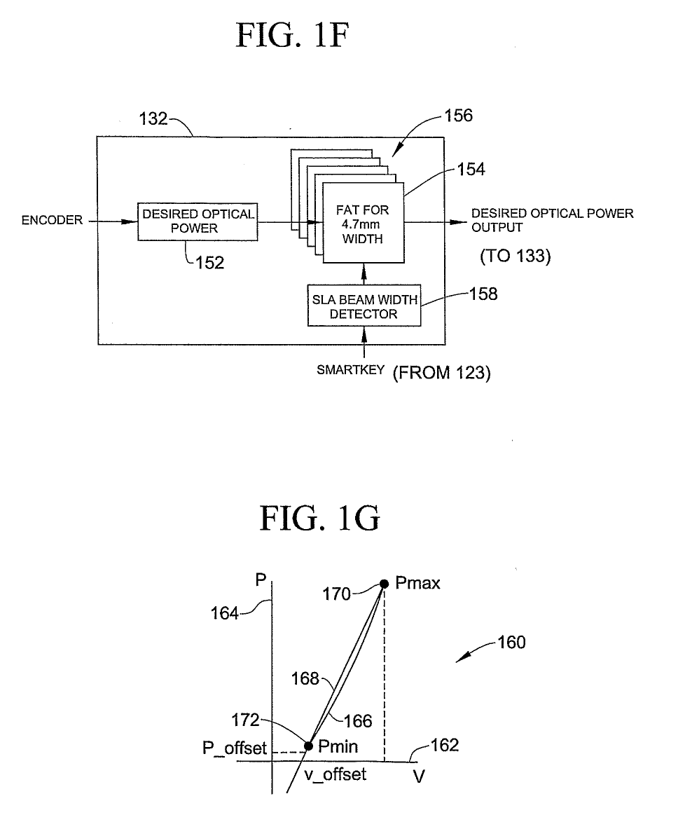

The laser system illustrated in FIG. 1A is an example of a photo-coagulator laser system and may be used in the application of a therapeutic procedure such as that described in WO 02/094260 “New use of ICG as a Photosensitive Agent,” published on 28 Nov. 2002.

The photo-coagulator laser system includes a photo-coagulator laser unit 10 followed by an optical delivery path. Upon exiting the laser unit 10, the laser beam travels through the optical delivery path, which prepares and delivers the laser beam to a delivery point at a distal end of the optical delivery path. During treatment the delivery point is applied to the patient's eye 100. The optical delivery system generally includes fibre optic cable 20, slit lamp adaptor 30, slit lamp microscope 40, beam splitter 50, and a delivery end (contact lens 60). The contact lens 60 (during treatment) usually contacts the area of the eye that requires treatment, and allows the laser beam to pass through to the eye. Other types of optical delivery path may be used, including an endo-ocular probe, a laser indirect opthalmoscope and a surgical microscope adapter.

FIG. 1B shows an overview of a laser system incorporating an auto-calibration system. Detector 70 is placed behind the contact lens 60 so as to measure the power of the laser beam at the end of the delivery path. Detector 70 converts the measure of the power of the laser beam to an electrical signal which is then fed via communication link 71 to an input 11 of laser console 10. This electrical signal is converted into a digital signal (unless the signal is already a digital signal) which is then provided to a processor in laser console 10.

The measurement of the power of the beam made by the detector at the delivery end is then compared with the desired or required power level for delivery. This information is used to adjust a calibration factor associated with the optical delivery path. The calibration factor is used in controlling the power of the laser beam generated by laser console 10. Accordingly the power generation compensates for the effect of the optical delivery path.

This allows the power of the generated beam to be controlled to provide the desired laser power level to the patient, even though the optical delivery path may vary significantly for different procedures.

The auto-calibration also accounts for power deviations caused by component variation and degradation in the delivery path, as well as within the laser console itself.

The laser system calibration method is carried out at the practitioner's discretion, but preferably prior to use for each patient. In one arrangement the laser system locks to prevent more than ten procedures being performed without an auto-calibration. Once the laser system has been calibrated, the detector 70 is removed from the delivery point to allow treatment of the patient's eye 100.

Generally, deviations in transmission factors of the delivery system result in a loss of power of the laser beam, however if the laser system is calibrated to account for a loss and, for example, the laser system components are cleaned or replaced at a later stage, then the power of the laser delivered at the delivery end can become greater than that calibrated for, resulting in possible injury to the patient. Regular calibration avoids such problems.

It is preferred in this regard, for example, for there to be about 8% or less variation in the power level of the laser delivered by the systems and methods disclosed herein. In particular, for example, it is preferred for there to be about 7% variation in power level or less, about 6% variation in power level or less, about 5% variation in power level or less, about 4.5% variation in power level or less, such as about 4% variation in power level or less, about 3.5% variation in power level or less, about 3% variation in power level or less, about 2.5% variation in power level or less, about 2% variation in power level or less, about 1.5% variation in power level or less, about 1% variation in power level or less, about 0.5% variation in power level or less, or even about 0.2% variation in power level or less.

As detailed in the embodiments disclosed herein, the i-MP system provides one or more of the following advantages: additional safety, higher efficacy, reduction in medication error, fewer side effects, less damage to a healthy retina, and is suitable treatment for all kinds of CNV secondary to AMD, Angioid Streaks, Pathologic Myopia, Central Serous Retinopathy, and other choroidal diseases resulting from inflammatory conditions and idiopathic causes as compared to conventional systems. In contrast to the method and system disclosed herein, conventional systems were operated in a substantially manual fashion. Typically, calculations needed to determine treatment parameters were performed manually and the person performing the treatment had to manually control the steps and timing of the procedure. Such systems were more susceptible to error. The present i-MP system reduces surgical and/or medication error that can adversely affect the patient since they are more precise than conventional systems and requires less operator intervention and/or discretion. The i-MP system also reduces surgical and/or medication error that can adversely affect a group, sampling, and/or population of patients since the systems and methods herein are more precise than conventional systems and requires less operator intervention and/or discretion.

Specifically, the treatment parameters that the operator is required to provide in certain embodiments of the i-MP system (for example, weight, lesion size, and pigment level) are easier to determine. In contrast, conventional lasers systems require the operator to determine parameters such as laser power, laser duration, photosensitizing agent, such as ICG, dosage, and laser spot size. The i-MP system herein requires the operator to make fewer adjustments, or in some embodiments no adjustments, to the laser power and laser duration. Furthermore, based on easily measured clinical parameter (for example, weight) the i-MP system will select for the operator the photosensitizing agent dosage and laser spot size to use. Therefore, the patient is subjected to fewer opportunities for operator error using the i-MP system herein.

As discussed herein, an error in these calculations may cause over or under treatment of the patient which may result in greater chance for unwanted side effects, damage to the patient's retina, and/or an ineffective treatment.

The automation introduced by the methods and systems herein create an added level of safety, require a reduced number of calculations and less control to be performed by hand, and furthermore, the parameters that are measured by the operator are simpler to determined (for example, lesion size, weight, and pigmentation level). Therefore, the present invention may allow for a broader range of physicians/operators with varying levels of skill and experience to treat patients and these treatments may generally be more effective as a whole. Furthermore, the present invention also allows doctors or operators to perform the disclosed treatments without having to use specialized diagnostic equipment such as ICG angiography imaging systems and similar equipment.

Before, when doctors used the i-MP system to treat patients without the control system herein, they would typically require formal supervision and training by a doctor who was experienced with the treatment procedure. The i-MP system herein reduces or eliminates this additional cost factor and allows a wider range of doctors or operators to perform the disclosed treatments. Using the i-MP system herein, doctors can perform the disclosed treatments using i-MP without having specific clinical training for these procedures and the supervision of another doctor. The i-MP system herein reduces the need for supervision, training, and provides a system that is much easier for a wider range of doctors or operators to use. Much of the decisions that a doctor would have to make with conventional i-MP systems are now subsumed in the automation that comes with the present invention.

Embodiments of the present invention use the photo-activation of Indocyanine Green (ICG) in the targeted tissue by the application of a continuous low irradiance laser to achieve selective vascular occlusion with minimal or no damage to adjacent neural structures or tissues. The dose of ICG and the dosing regiment can be varied if desired. The total dosage of ICG used per treatment session is typical either about 100 mg or about 150 mg based upon the weight of the patient. In certain embodiments, the total dosage can be varied to about 60 mg to about 170 mg per treatment session, about 70 mg to about 160 mg per treatment session, or can be about 90 mg, about 95 mg per treatment session, about 105 mg per treatment session, about 110 mg per treatment session, about 140 mg, per treatment session about 145 mg per treatment session, about 155 mg per treatment session about 160 mg per treatment session. The solution concentration used can also vary. In certain preferred embodiments the concentration will be about 25 mg/ml. This can be varied either as more concentrated or less concentrated if desired. In certain embodiments the solution could range from about 10 mg/ml to about 40 mg/ml, about 15 mg/ml to about 35 mg/ml, or about 20 mg/ml to about 35 mg/ml. Typically in certain embodiments each subject received a 2 ml loading dose of a solution of ICG (25 mg/ml, approximately 1 mg/kg body weight) in the form of an IV bolus, followed by a 5.0-ml saline flush. 20-40 minutes after the loading dose, a second IV injection of 50 mg of ICG in 2 ml of solution is administered to each subject, followed by another saline flush. In the past ICG has typically been provided as a powder in a tube in the amounts of 25 mg and 50 mg per tube. This has required use of multiply tubes which added to the potentially for medication error. In some embodiments of the present invention, the ICG powder is provided as a freeze dried powder in a tube so the solution can be mixed in one tube and used as one dose amount. Embodiments of the present invention contemplate providing the ICG as a freeze dry powder in 5 ml tubes with 100 mg ICG and in 10 ml tubes with 150 mg ICG. It is believed providing the dosage to be used in a single vial will contribute to the reduction of medication error. Of course the amounts of ICG and the size of the tubes could be varied.

The i-MP system herein includes varying of degrees of automation in the computer-implemented method of controlling a therapeutic procedure. Certain embodiments will be substantially automated whereas other embodiments will be less automated. The degree of automation may vary. One skilled in the art will appreciate that one or more of the steps, data determinations, patient-related data determinations, dosage levels, calculations, desired outputs, treatment parameters, displays, prompts, instructions, and timing issues disclosed herein to some extent can be done manually. Some embodiments permit varying degrees of automation. For example, in some embodiments, the dosage level of ICG will be either 100 mg or 150 mg based on the weight of the patient being treated. Some embodiments allow for the selection of the dosage to be performed manually. For example, in some embodiments the spot size could be manually determined by the doctor and manually selected by the doctor. For example, in some embodiments the doctor could use a stopwatch to manually control and prompt the thirty minute wait after the first injection of ICG to inject the second ICG dose, and/or the two minute wait to apply the laser after the second injection of ICG.

In certain embodiments, the percentage of successful treatments may increase by between about 1.05 to about 2 times, 1.25 and about 4 times, greater than about 1.05 times, greater than about 1.1 times, greater than about 1.2 times, greater than about 1.3 times, greater than about 1.4 times, greater than about 1.5 times, greater than about 1.7 times, greater than about 2 times, between about 1.05 and about 1.4 times, between about 1.25 times and about 1.6 times, or between about 1.3 times and about 2 times the current number of successful treatments.

In certain embodiments, the percentage of errors during treatment may be reduced by between about 20% and about 100%, greater than about 5%, greater than about 15%, greater than about 25%, greater about 35%, greater than about 50%, greater than about 75%, between about 5% and about 15%, between about 10% and about 25%, between about 10% and about 30% as compared with conventional treatments. As discussed above, the i-MP system herein allows a broader range of physicians/operators to treat patients. This is the result of several factors including, but not limited to, an increased level of comfort for physicians, reduction in the need for specialized training, reduction in the need of specialized diagnostic equipment such as ICG angiography imaging systems, optical coherence tomography (OCT) and similar equipment, and/or the ease of use of the automated systems and methods of the present invention as compared with conventional systems. In certain embodiments, by using the present invention, the percentage of physicians treating patients may increase by between about 1.1 times and about 4 times, greater than about 1.1 times, greater than about 1.2 times, greater than about 1.25 times, greater than about 1.4 times, between about 1.2 times and about 1.4 times, between about 1.3 and about 2 times, or between about 1.4 and about 1.8 times the current number of physicians treating patients.

In certain embodiments, by using the present invention, the percentage of physicians treating patients may be increased. If the population of ophthalmologist is defined as physicians who treat retinal diseases and you randomly select 10 of these physicians from a city or urban setting with a population of over 2 million people, then in certain embodiments, by using the present invention, the percentage of physicians treating patients may increase by between about 1.1 times and about 4 times, greater than about 1.1 times, greater than about 1.2 times, greater than about 1.25 times, greater than about 1.4 times, between about 1.2 times and about 1.4 times, between about 1.3 and about 2 times, or between about 1.4 and about 1.8 times the current number of physicians treating patients.

In certain embodiments, by using the present invention, the percentage of physicians treating patients may be increased. If the population of ophthalmologist is defined as physicians who treat retinal diseases but have treated less than 10 patients with AMD using available treatment modalities and you randomly select 10 of these physicians from a city or urban setting with a population of over 2 million people, then in certain embodiments, by using the present invention, the percentage of physicians treating patients may increase by between about 1.1 times and about 4 times, greater than about 1.1 times, greater than about 1.2 times, greater than about 1.25 times, greater than about 1.4 times, between about 1.2 times and about 1.4 times, between about 1.3 and about 2 times, or between about 1.4 and about 1.8 times the current number of physicians treating patients.

The above mentioned improvements that allow a broader range of physicians/operators to treat patients is the result of several factors including, but not limited to, an increased level of comfort for physicians, reduction in the need for specialized training, reduction in the need of specialized diagnostic equipment such as ICG angiography imaging systems, optical coherence tomography (OCT) and similar equipment, the reduced need for specific clinical training on i-MP and/or the ease of use of the automated systems and methods of the present invention as compared with conventional systems.

### Description of the Laser Console

In certain embodiments, referring now to FIG. 1C, there is shown a system overview of the application device, laser unit 10, which may be employed in a therapeutic procedure according to an illustrative embodiment of the present invention. In this illustrative embodiment, the i-MP treatment or diagnostic system is the i-MP procedure described previously.

While this illustrative embodiment is described with reference to the i-MP procedure, the described arrangement may be applied to other medical procedures involving an application device used with externally introduced substances which when used together facilitate the medical procedure.

Application device 10 includes a control system 90, laser assembly 80, slit lamp adaptor (SLA) 30, display 117, keyboard 116 and foot pedal 121. Laser assembly 80 is a laser photocoagulator system which delivers controlled pulses of continuous wave 805 nm wavelength laser. The laser assembly 80 can deliver a maximum of 2.5 W of power which is continuously monitored by redundant safety systems. As illustrated in FIG. 1D, the laser console 10 includes a laser, a laser power supply, an electronics control board, an electronics power supply board, a control panel with display, keypad and buttons, a control panel board and a microcontroller board. Although the embodiment of the laser assembly described herein has a maximum power of 2.5 W, it will be readily understood that any laser assembly with similar power specifications (e.g., about 2.5 W, less than about 3 W, less than about 5 W, about 2.75 W, about 2 W, about 1.5 W) could be used as well.

SLA 30 performs the function of delivering the laser beam to the patient's eye. It is an optical device including a fibre optic cable, a Galileo type microscope and a mechanical system which permits the device to be attached to a slit lamp microscope. SLA 30 is positioned coaxially with the optical path of the slit lamp microscope and allows the physician to apply the laser whilst viewing the back of the patient's eye (retina).

In one arrangement control system 90, keyboard 116, display 121 and laser assembly 80 are integrated into the same enclosure. Control system 90 is a microprocessor-based electronic circuit which runs the operational software and is responsible for controlling the operation of the laser assembly 80, aspects of the laser safety monitoring and interaction with the user interface including keyboard 116, display 117, user controls and foot pedal 121. Control system 90 also runs the routines which control the delivery of the treatment procedure. Control system 90 includes a microprocessor, memory, software, power supplies and other related electronics.

FIG. 1D shows the laser console 10 in greater detail and illustrates the system components included in the laser assembly 80 and control system 90. The main laser power supply 101 supplies the required current to produce the laser beam. The main laser power controller 102 is a module that controls the current to the main laser so that the output power is equivalent to the desired power. The laser diode 103 is used to generate the laser beam for the procedure. The wavelength of the laser is 805 nm, which is in the infrared range and is invisible to the human eye. The laser preferably has a tolerance of +1-3 nm. The laser produced by diode 103 passes through the main laser collimator lens set 104, which shapes the laser beam so that the beam can be focused onto the fibre-optic cable.

After the lens set 104, the beam passes through beam splitter 105, which is a partially reflective mirror that splits the laser beam, providing a percentage of the laser beam to a photo sensor 112 that forms part of a safety system.

The part of the beam that is not diverted by the beam splitter 105 reaches the aiming beam combiner 106, which is a special mirror that combines the main laser beam from diode 103 with an aiming laser beam received from laser diode 113. The aiming laser beam has a visible beam (red) that is used by the physician to aim the laser. In one arrangement the aiming beam laser has a wavelength of 630 nm and a maximum power of 1 mW. In contrast, the main beam has a maximum power of 2.5 W.

After the aiming beam combiner 106, the combined beam passes through a fibre coupler lens set 107 that focuses the laser beam onto the fibre optic cable of the optical delivery path.

Laser cavity 111 is a metal box which contains the main laser diode 103, and the optical components 104, 105, 106 and 107 used to adjust the shape, focus and direction of the laser. The aiming laser diode 113 may also be included in the laser cavity. The optical delivery path 110 is connected to an output nozzle of the laser cavity 111. The cavity 111 is sealed to protect the optical system from dust and humidity. At the output nozzle of the laser cavity 111, there is an optically-coupled fibre lock sensor 108 that indicates to the controller whether there is a fibre optic cable connected to the laser console 10. A mechanical laser shutter 109 is connected by a hinge to the laser console 10 to cover the output nozzle when no delivery device is connected to the laser console 10.

The laser console 10 may be connected to an optical delivery path 110 which includes a fibre optic cable used to deliver the laser beam to the patient's eye. Examples of optical delivery paths include an endo-ocular probe, a slit lamp adaptor, a laser indirect opthalmoscope and a surgical microscope adapter.

Some of the beam split by beam splitter 105 is provided to the main laser safety photo-sensor 112, which is a photodiode that reads the power level and provides an electronic signal used to ensure safe laser operation.

Processor 114 controls the functioning of all the laser equipment, and is in electronic communication with most of the components of the laser console 10. In one arrangement the processor 114 includes a microprocessor from the 8032 family, flash memory, e2prom and a watchdog unit. The processor 114 has access to data storage in which parameters of the therapeutic procedure may be stored. A buzzer 115 connected to the processor 114 is used to generate alarms, beeps and other audible signals.

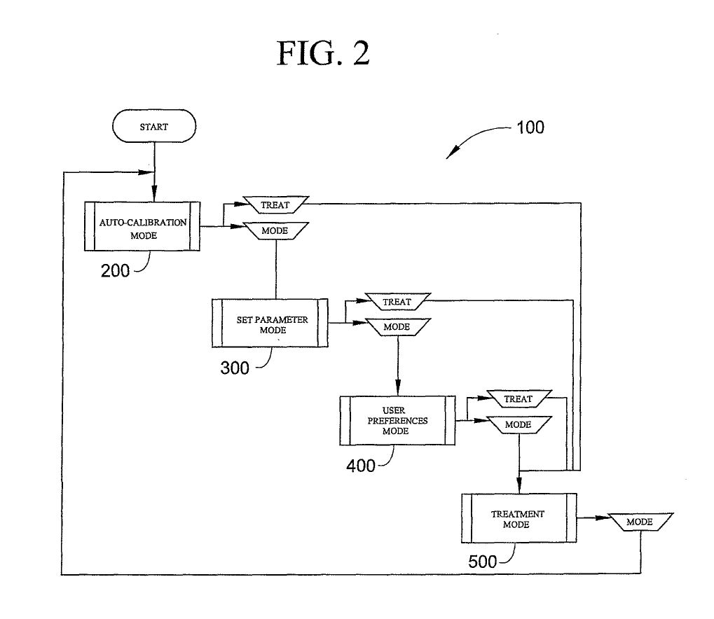

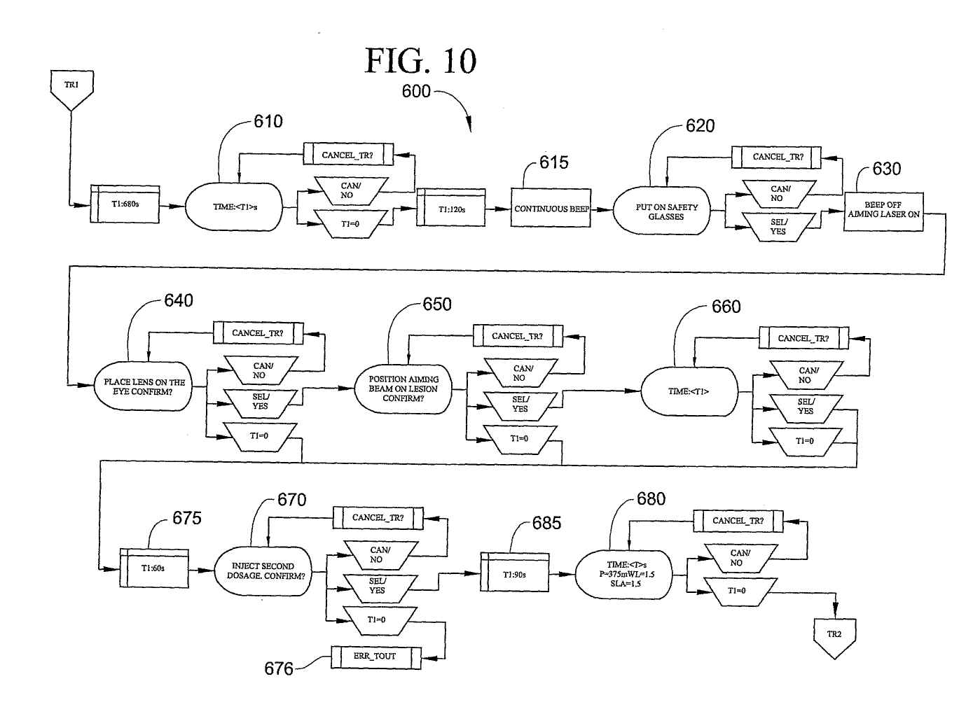

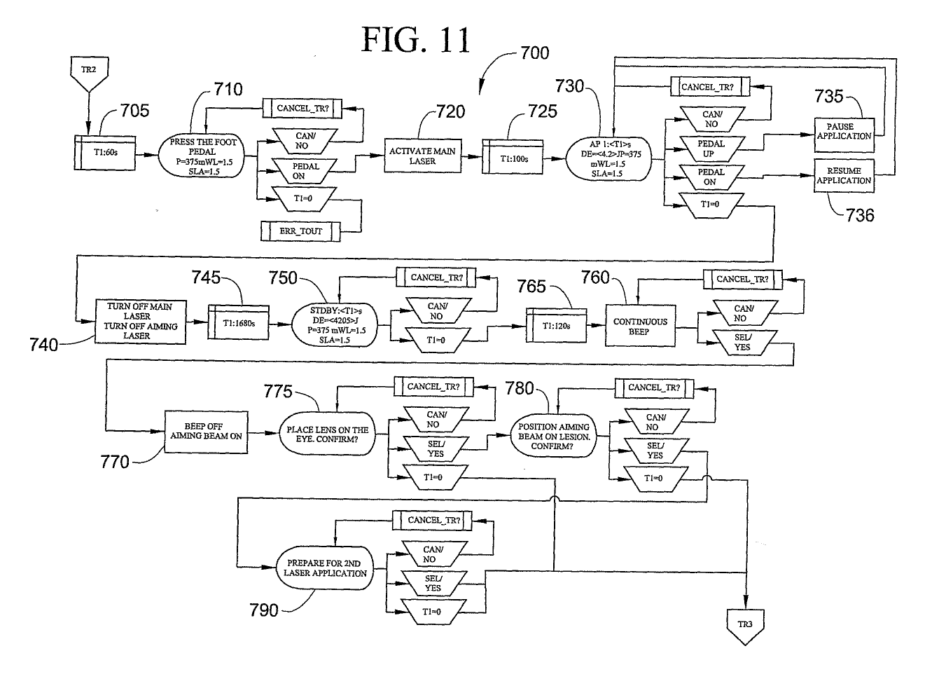

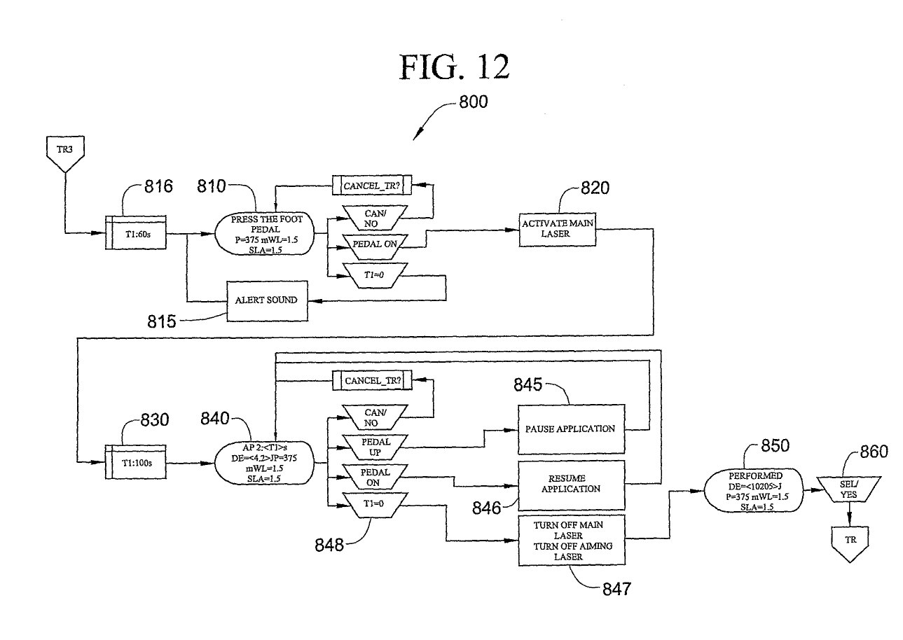

Keyboard 116 is used as an interface for the physician or operator to control the operating mode and parameters of the treatment, and the alphanumeric display 117 is used as an interface to show the treatment data and parameters to the physician using the laser console 10. As described in more detail with reference to FIGS. 2 to 13, the visual and audio outputs of the laser unit 10 may be used to guide an operator through the i-MP procedure.

A laser power knob 118 is preferably a rotary knob allowing the physician to set the main laser power. The power knob includes an encoder from which output signals are read and interpreted by the processor 114 and displayed to the physician.

The pulse-duration-select dial button 119 is a rotary knob allowing the physician to set the duration of a laser shot. The button 119 includes an encoder from which output signals are read and interpreted by the processor 114 and displayed to the physician, for example, on display 117.

The pulse interval select dial button 120 is a rotary knob which allows the physician to set the repeat interval. Diode button 120 includes an encoder from which output signals are read and interpreted by the processor board 114.

Foot switch 121 is used to fire the laser beam. The foot pedal 121 is optically coupled to the laser console 10 to provide electrical safety.

Interlock unit 122 is an optional device for additional laser safety. The interlock input 122 allows a switch to be connected to the laser console 10 to disable the laser when an external door is opened inadvertently. If the user chooses not to use the remote interlock, then a by-pass connector must be inserted into the interlock unit 122 to enable operation of the laser.

The “autokey” connector 123 contains electrical circuitry used to provide information to the laser console 10 that indicates what optical delivery path has been connected to the laser console 10. Each optical delivery path 110 has different transmission properties which affect the laser power that reaches the patient's eye 100. Information provided to the laser console 10 via the autokey connector 123 enables the console 10 to recognize the delivery device in use so that the processor 114 can calculate a transmission factor (FAT) to compensate for the attenuation of laser power along the optical delivery path 110.

An electronic power supply 124 supplies the required power to the circuits of the power controller 102 and the processor board 114. EMI/EMC line filter 125 is a module that filters the electrical noise from the mains line to protect the laser from malfunction and damage due to possible power surges. Mains cable 126 connects the laser console 10 to an electric outlet. Switch 127 is an on/off switch allowing the user to turn the laser console 10 on or off.

Keyboard 116 includes a number of buttons for the operation of application device 10 and adjustment of the treatment parameters by an operator. The buttons include:

| | |
|---|---|
| <Treat> | Activates TREATMENT mode directly; |
| <MODE> | Used to select the instrument's operating mode; |
| <SEL/YES> | Selects or accepts the displayed option; |
| <INC> | Increments the selected parameter; |
| <DEC> | Decreases the selected parameters; |
| <CAN/NO> | Cancels or declines the displayed option, and if |
| | pressed for some time, cancels the ongoing process; |
| <emergency> | Emergency button - aborts all operations and |
| | places the device in emergency interruption mode. |
| <pedal> | Foot pedal. |

Referring now to FIG. 1E, there is shown a flowchart diagram of a power control system 130 for use with the laser console 10. Processor 114 controls a safety circuit 144 that sends a signal to actuator 136 to turn off the laser diode 103 if a fault is detected, thus preventing the laser from firing a shot if the power level is not in specified limits. The processor 114 is a unit where operational software is stored and executed. When a command is received to activate the laser, a reference block 132 generates a reference signal that relates to the level of power desired at the delivery point at the end of the optical delivery path 110. The reference block 102 may be a module of the processor 114.

The reference signal provided by reference block 132 is converted to an analogue voltage by the D/A converter 133. In turn the analogue signal is provided to subtraction block 134. The subtraction block 134 has another input signal that corresponds to the amount of power that the laser diode 103 emits in the laser cavity. The subtraction block 404 compares its two inputs to generate an error signal that is provided to PID controller 135. The input signal of the PID controller 135 is thus the difference between the desired power and the actual power generated in the laser cavity 111. The PID controller 135 amplifies the error signal, taking into account the dynamics of the system, and sends the amplified signal to the actuator 136 which directly controls the current to the laser diode 103.

Dual photodiodes 112 monitor the output of the laser diode 103 to provide feedback signals for the power control and safety functions. One of the photodiodes 112 sends a voltage signal corresponding to the power level in the laser cavity 111 to the subtraction block 134. The other photodiode 112 sends a voltage signal to the A/D converter 143 which provides a digital signal to the processor 114 indicative of the actual power level. The signal that passes via A/D converter 143 is not used in the power feedback loop but instead is used in the safety circuit 144. If the power in the laser cavity 111 exceeds the set power by more than 20%, the laser diode 103 is switched off immediately and an error message is displayed on the alphanumeric display 117.

As the laser beam is transmitted through the optical delivery path, the beam may be attenuated and some laser power is lost in the transmission. It is necessary to estimate the attenuation of each optical delivery path and spot size and to use this information in the power control of the laser console 10. For example, if SLA 30 with a selected spot size of 200 micrometers has an estimated attenuation of 20%, the power generated in the laser cavity 111 must be increased by 20% so that the power that hits the patient's eye 100 matches the power set by the physician.

The amount of attenuation is noted at the factory during production of the optical delivery path. Based on the attenuation a correction factor is calculated, the transmission factor (FAT), also referred to as the compensation factor. The transmission factor is recorded in the memory of the laser console 10 for each type of delivery path and spot size for which the laser console 10 is used. The transmission factor is used by the laser console 10 to adjust the power when using an SLA 30. The same system is used for other delivery devices, although endo-ocular probes and laser indirect ophthalmoscopes have only one fixed spot size. The SLA 30 used for the i-MP procedure has a magnification changer which produces five different laser spot sizes. In one arrangement the spot sizes are: 0.8 mm, 1.0 mm, 1.5 mm, 2.5 mm and 4.3 mm in diameter at the focal point of the SLA 30. In some embodiments, the SLA may have has a continuous, or substantially continuous magnification zoom system with spot sizes ranging over the desired mm range. In some aspects, the zoom system spot size may range from 0.5 mm to 5 mm, 0.25 mm to 6 mm, or 0.5 mm to 4.5 mm. In certain embodiments, the spot sizes will be selected from the following ranges: 0.7 mm to 0.9 mm, 0.95 mm to 1.0 mm, 1.25 mm to 1.75 mm, 2.25 mm to 2.75 mm and 4.1 mm to 4.5 mm in diameter at the focal point of the SLA 30.

For each spot size selected, the laser beam passes through a different lens set resulting in different attenuation of the beam for each spot size. In order to compensate for the attenuation, the laser console 10 must be informed of the selected spot size so that the correct transmission factor (FAT) is used in the calculations.

FIG. 1F illustrates the operation of reference block 132 for adjusting the power for different spot sizes. Reference block 132 may be implemented as a sub-system of the processor 114. Block 132 receives an input from the autokey 123 that enables slit-lamp-adaptor beam-width detector 158 to recognize the type of optical pathway in use and the selected spot size. Detector 158 is thus an optical path identifier. Block 132 uses this information to select an appropriate FAT (for example the FAT 154 for a spot width of 4.7 mm) from a set of transmission factors 156 stored in memory. Another input to block 132 enables the physician to specify the desired optical power, for example by means of power knob 118. Block 132 multiplies the desired optical power 152 by the selected FAT 154 to produce a desired optical power output, which is presented to the D/A converter 133.

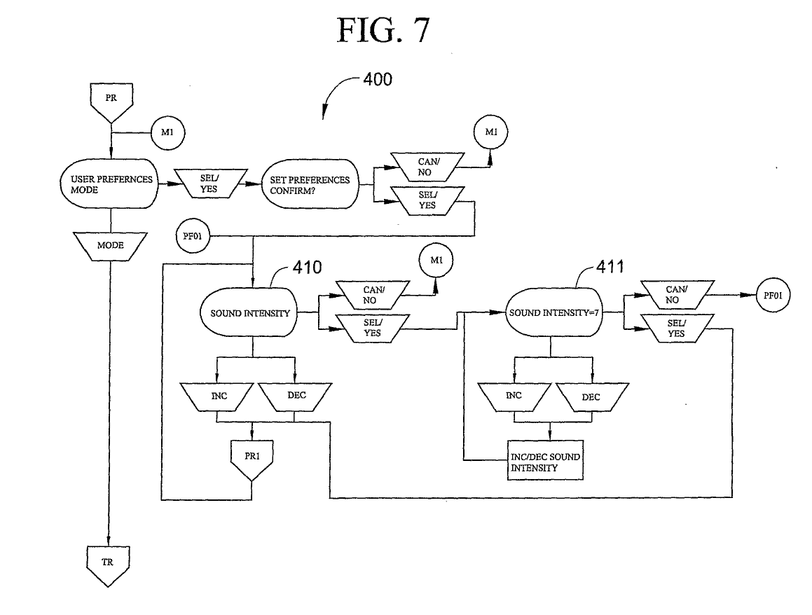

As a result of this control system, the power delivered to the patient's eye 100 is theoretically equivalent to the power set by the physician for any power level. If the parameters of the PID controller 135 are well selected, there should be no oscillations of power generated by the laser diode 103. However, there are many factors that can affect the power delivered to the patient's eye which cannot be detected by the system shown in FIG. 1F. The auto-calibration device shown in FIG. 7 addresses some of these limitations and increases the precision of the power control.

FIG. 1G illustrates the power output of the laser diode 103 in response to a given reference voltage. The graph 160 shows power 164 versus voltage 162. The ideal response 168 of the photo diode 103 is linear between a minimum point 172 and a maximum point 170. In practice, the actual response code 166 is non-linear.

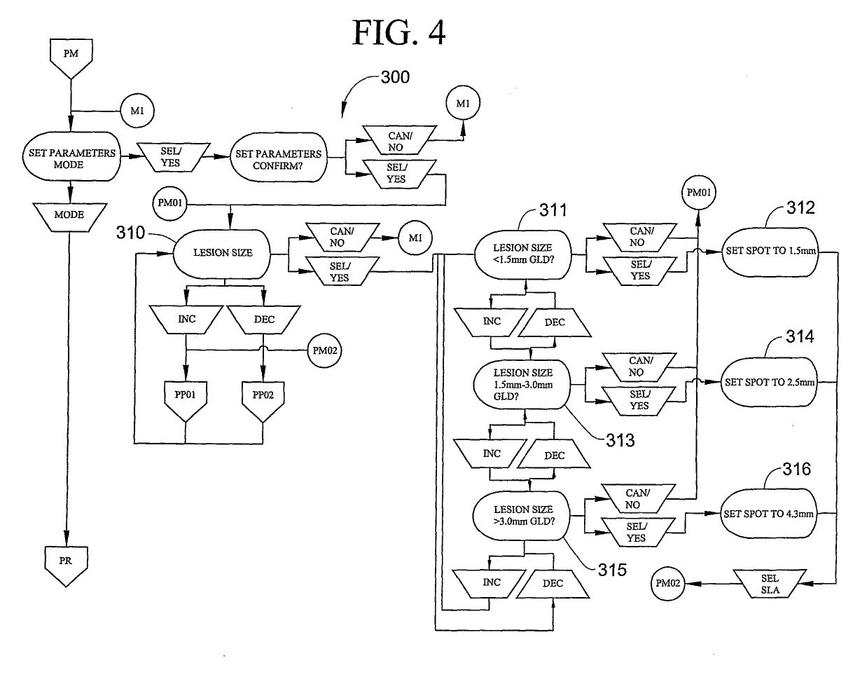

Errors in power transmission may be caused by the fibre optic coupling. The fibre optic couplings include high precision connectors where the physician or operator inserts the delivery devices via the optic cable into a receiving port and twists the fibre optic clockwise until the end connector reaches the end of the course. However, a small shift in position can be caused by simply removing the fibre optic cable and inserting it back into the port. This can cause an error of up to 5% in the transmitted power. Since the power controller 130 operates within the laser cavity 111, this power error is not detected by the system and is therefore not corrected for. In addition, any type of dust or dirt on the lenses of SLA 30 can cause attenuation in the power delivered by the system. Again, because this happens outside the laser cavity 111, the error is not corrected by the system of FIG. 4.

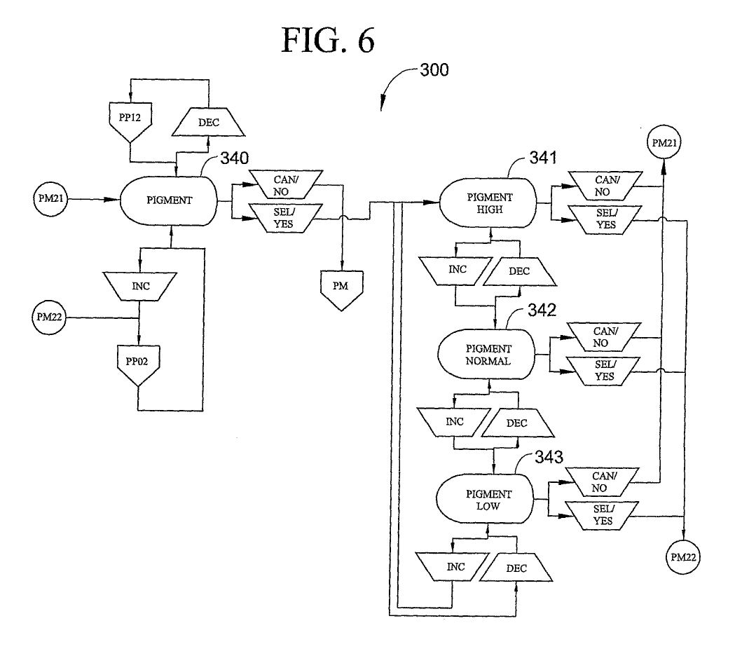

Aging of the laser diode 103 is another factor causing error in the power control system. During factory calibration the laser diode 103 presents a characteristic curve, for example that shown in FIG. 6. The system is then calibrated between the minimum point 172 and maximum point 170 so that within the dynamic range of the laser diode 103 the error is the smallest possible. As the diode 103 ages, the shape of the curve changes and consequently the minimum and maximum points may shift. If a laser diode 103 is only calibrated during production, this error tends to increase over time as the laser is used.

Other factors contributing to error in the system include the appearance of microfissures in the fibre optic cable or a misalignment of the fibre coupling.

### Selecting the Mode

In certain embodiments, referring now to FIG. 2, there is shown a flowchart diagram of the mode selection process 100 of the treatment system. The treatment system has four modes of operation including:

AUTO-CALIBRATION mode 200: This mode is selected to calibrate the output power of laser unit 10. This calibration is necessary due to the precision required for the i-MP procedure. Auto-calibration is designed to compensate for any output power deviation arising either from accumulation of dust on the mirrors and lenses of the SLA 30, wearing out of the fibre optic, misalignment or aging of the laser diode 103. The adjustment range of the auto-calibration is 20% of the factory calibration thereby preventing the accidental use of the equipment out of the power tolerance specification. In some embodiments, auto-calibration may not be performed before each procedure. In some embodiments, the calibration may be a manual procedure or it may be performed after, for example, every two procedures, every five procedures, or every ten procedures. Additionally, in embodiments, the adjustment range of the auto calibration may be about 20% (e.g., about 15-20%, about 10-20%, about 10-30%).

SET PARAMETER mode 300: This mode includes a sequence of screens displayed on display 117 where the user is prompted to adjust the fundamental parameters of the treatment procedure including:
Lesion greatest linear dimension (GLD);
Patient's weight;
Lens magnification; and
Pigment concentration.

USER PREFERENCES mode 400: In this mode auxiliary parameters such as aiming beam intensity and sound intensity of buzzer 115 are adjusted by the operator.

TREATMENT mode 500: Mode in which the treatment laser 103 is applied to the patient using previously selected parameters.

Mode selection is accomplished by the operator pressing the <MODE> button repeatedly until the desired mode is displayed on display 117. Once a mode is shown on the display, pressing the <SEL/YES> button will commence the associated sequence of steps to be performed for that mode.

The procedures of FIGS. 2-12 are performed by software running on processor 114. Prompts are displayed to the operator on display 117 and the operator interacts with the software by pressing a button on keyboard 116 or pressing foot pedal 121. In some instances the operator is prompted to perform an action, for example putting on safety goggles or injecting the patient. The software procedure in general does not proceed until the operator has confirmed (by pressing a button on keyboard 116) that the action has been performed. For ease of description, the following text does not mention every point in the software where the operator is required to confirm that he or she wishes to proceed to the next step.

### Auto-Calibration

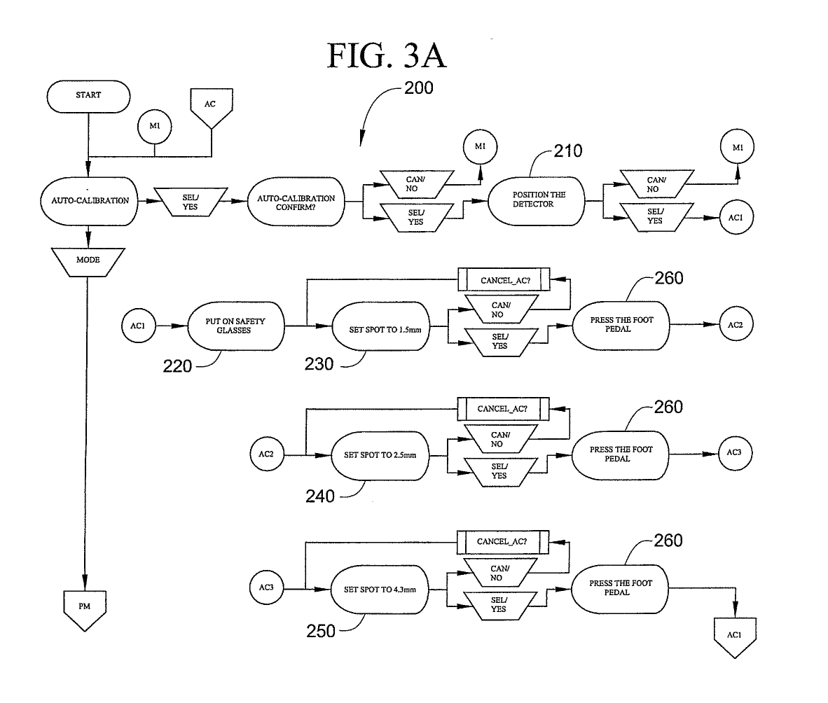

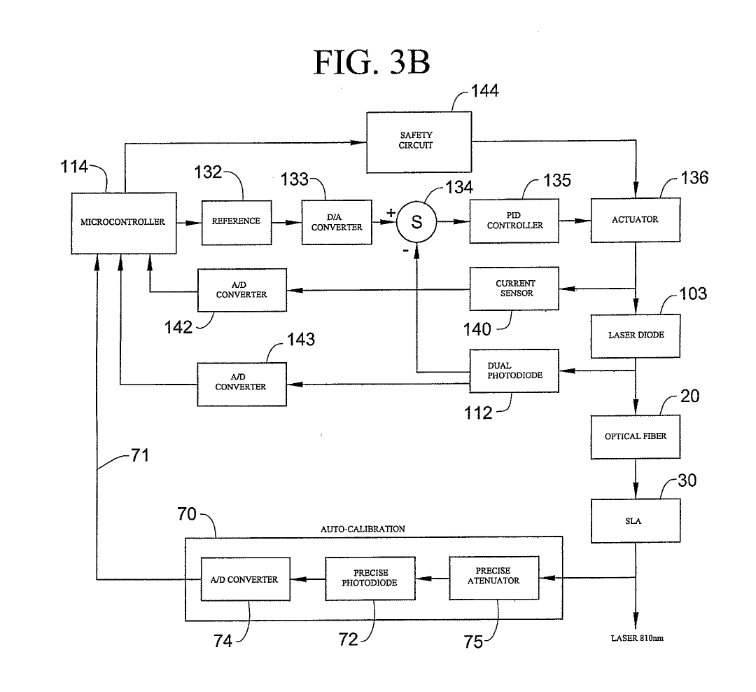

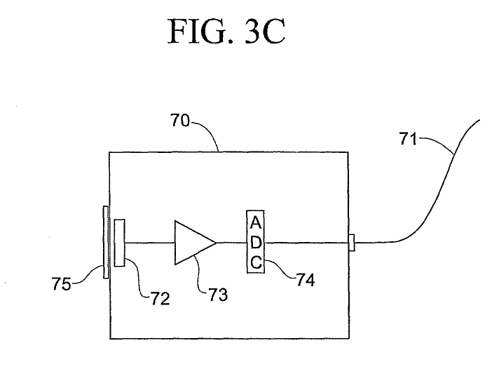

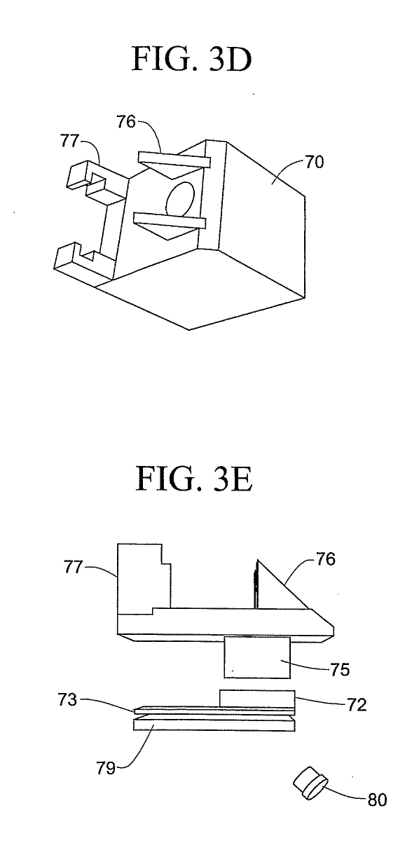

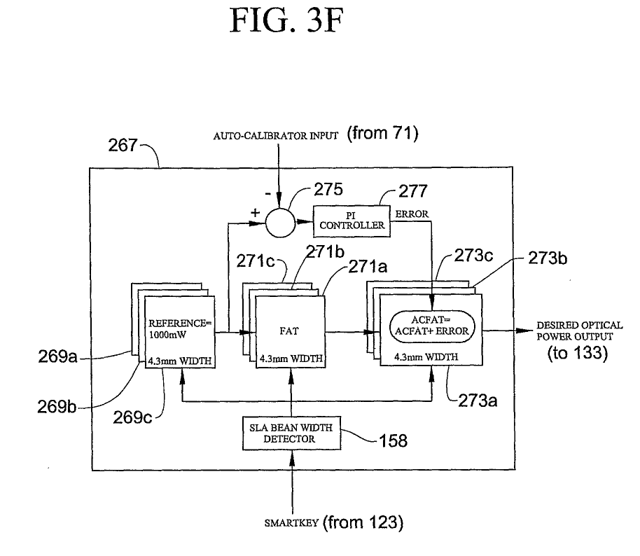

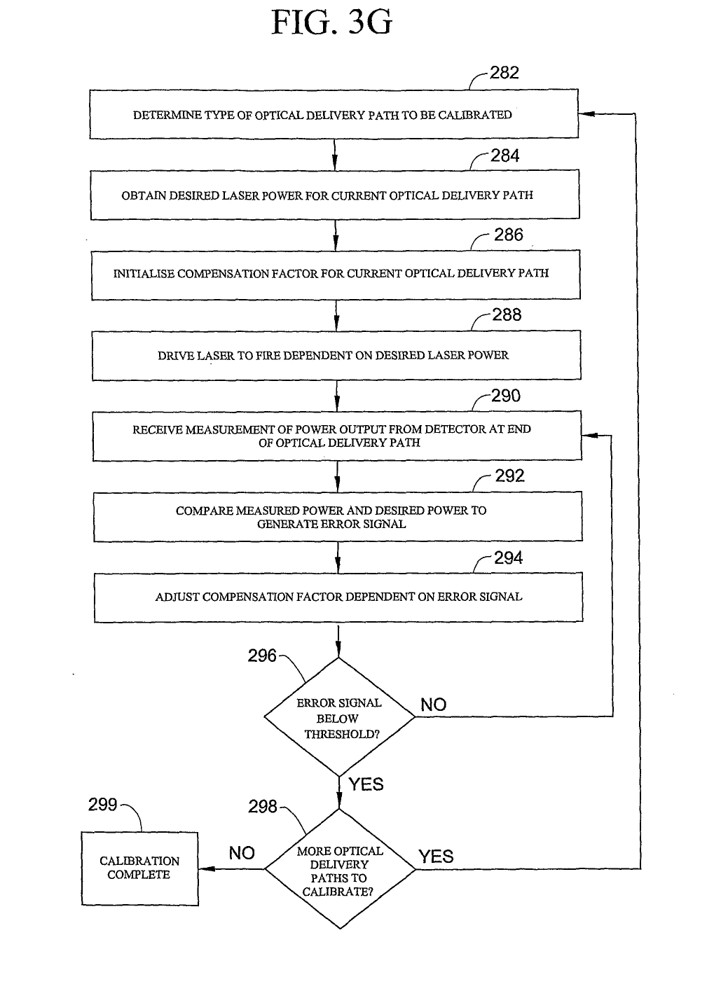

Referring now to FIG. 3A, there is shown a flowchart diagram of the steps involved in guiding an operator through the auto-calibration of laser unit 10 with a particular optical path in place. AUTO-CALIBRATION mode 200 is used to fine tune the system's power control and compensate for any degradation and aging of the components. Dust on the mirrors, lenses and filters, micro-cracks in the fibre optical cable or misalignment of the fibre optic coupling are the most common causes of deviation of the output power of the laser. Additionally, the laser diode also ages and although this is somewhat compensated for by an internal closed-loop circuit there may be laser degradation to an extent that cannot be compensated by this circuit resulting in the requirement for external adjustment.

Application devices such as laser unit 10 are also governed by various standards which seek to ensure the safety of medical equipment. These standards stipulate that the power control must not exceed 20% of deviation. However, as the i-MP process is critically dependent on the irradiance of laser assembly 80, more accurate control down to the 5% level is required. The auto-calibration process involves the use of a purpose-designed power meter 70, which is placed in a position that corresponds to the location of the patient's eye 100 to measure laser power. The auto-calibration procedure is automatic, however the operator is required to position the power meter, connect the power meter cable 71 to the input 11 of the laser console 10 and activate the auto-calibration routine. As shown in FIG. 3, the laser console 10 prompts the user to perform the necessary actions. In some embodiments, a commercial off-the-shelf power meter may be replaced for power meter 70.

To select AUTO-CALIBRATION mode 200 the operator presses <MODE> button on keyboard 116 repeatedly until display 117 shows the message:

<MODE> Auto-calibration.

The operator then confirms that he or she wishes to complete the auto-calibration procedure and is then prompted 210 to position the power meter or detector 70 at which point the software running on processor 114 activates the aiming laser diode 113 to assist in the positioning.

After the operator has confirmed (by pressing the CAN/NO or SEL/YES buttons) that the detector 70 is positioned, the controlling software then prompts 220 the operator to wear his or her safety glasses before proceeding with the auto-calibration procedure. The first part of the procedure involves setting 230 a spot size to 1.5 mm. This is performed manually by the operator turning the thumb wheel on the SLA 30 to the 1.5 min position at which point the operator is prompted 260 to either cancel the auto-calibration or press the foot pedal 121 thereby activating the laser diode 103 which will be fired for a period of time long enough to complete the internal calibration performed by software running on processor 114. The laser will be turned off and the user will then be prompted 240 to adjust the spot size to 2.5 mm and repeat the laser firing procedure. The software then prompts 250 the operator to adjust the spot size to 4.3 mm and once again activate the laser by pressing the foot pedal (prompt 260). In some embodiments, calibration may be performed using other spot sizes for example, 0.8 mm, 1.0 mm, 1.5 mm, 2.5 mm, and 4.3 mm.). It is also possible to have systems that have a continuous spot size adjustment that are sometimes referred to as zoom magnification systems. It is also possible to have other spots sizes selected from the range of 0.5 mm to 6 mm.

Depending on the results determined using the power measured by the detector 70, the operator will be informed of a successful calibration procedure or alternatively in the event of failure be prompted to take remedial action such as replacing the fibre 20 and/or cleaning the optics at which stage the auto-calibration procedure can be repeated.

In auto-calibration mode, the power control system adds a further compensation factor (ACFAT), which is generated by the auto-calibration system. The desired power selected by the physician is multiplied by the FAT and by the ACFAT to generate the reference voltage presented to the D/A converter 103.

FIG. 3B is a flowchart similar to the flowchart of FIG. 1E, with the addition of detector 70 that reads the laser power at the delivery point of the optical delivery path and provides an electrical signal indicative of the laser power back to processor 114. Detector 70 includes a precise optical attenuator 75, a photodiode 72 to measure the incident power, and an A/D converter 74 to provide a digital signal that may be fed back to processor 114 via communication link 71. The attenuator 75 attenuates the incident laser power to the operational range of the photodiode, so that the photodiode 72 does not saturate or get damaged.

For each power range to which the power of the laser console 10 can be set, there is a specific ACFAT that is calculated every time an auto-calibration routine is executed. When auto-calibration is completed, the laser control system returns to its normal operating mode as illustrated in FIG. 4.

FIG. 3C shows a diagram of detector 70 which contains a high-precision photodiode 72, and converts the power measurement of the laser received by photodiode 72 into an electrical signal, which is then amplified by amplifier 73 and converted into a digital signal by ADC 74. This signal is then transmitted into laser console 10 via link 71. The ADC conversion may alternatively be performed within laser console 10 itself, or any other convenient location. Detector 70 has an optical filter 75 which blocks light of visible wave-length allowing only the near infrared wave-length to reach the photodiode 72. The filter 75 attenuates the laser power to prevent saturation of the photodiode 72.

FIG. 3D shows a particular detector 70 designed to be attached to a slit lamp adaptor through a mechanical coupling system 76 and 77. Detector 70 and the body of the SLA 30 have marks to guide the appropriate positioning of the detector 70. Before commencing the auto-calibration, the physician or operator attaches the detector 70 to the body of SLA 30 assisted by mounting posts 76 and guiding posts 77.

FIG. 3E shows the components of the detector 70 including laser beam attenuation filter 75, photodiode electronics circuit board 73, precision photodiode 72, electronic circuit board fixation mounts 79 and a power supply and signal cables protecting boot 80.

In one arrangement, the adjusting margin of the auto-calibration is approximately 3% of the factory setting. This prevents the use of the equipment out of the calibrated and nominal operational conditions. In one arrangement the system permits 10 treatments to be performed between calibrations. If the number of treatments exceeds 10, the laser console will self-lock and display a message to the physician requiring calibration, preventing the physician from performing a new treatment until a new auto-calibration has been successfully performed. It is recommended that an auto-calibration be performed every time the laser console 10 is not used for a period of 3-5 days, or when the delivery device has been disconnected.

The operator presses a mode button until the display 117 shows the message “auto-calibration mode”. The operator presses a select/okay button to enter this mode, following which the operator is prompted to confirm whether he or she wishes to enter the auto-calibration procedure. After confirmation, a message will prompt the user to wear safety goggles and confirm that goggles have been put on. The user then positions the detector 70 on SLA 30. The aiming laser may be automatically turned on to assist the user in positioning the detector 70. The next message displayed may ask the user if the marks at the detector 70 and SLA 30 are aligned. The operator may press a select/okay button to confirm. At this point the procedure has the user to select a spot size, for example 1.5 mm, and the system will display a message confirming when the thumb wheel setting the spot size is at the correct position. The system then prompts the user to fire the laser by pressing the foot pedal 121. As soon as foot pedal 121 is pressed display 117 shows calibration parameters to allow the user to monitor the calibration.

FIG. 3F is a flowchart of the functioning of laser calibrator 267 which replaces block 132 in FIG. 3B. Calibrator 267 receives an input from the autokey 123 which enables the beam-width detector 158 to detect which optical delivery path and spot size have been selected. Dependent on the selected spot size, laser calibrator 267 retrieves a FAT 271a-c corresponding to the selected spot size. The calibrator 267 also selects an ACFAT from the set 273a-c corresponding to the selected spot size and a reference input 269a-c corresponding to the selected spot dimensions. For example, for a spot width of 4.3, the reference input to be used in the calibration procedure may be 1000 mW.

The ACFAT 273a is initialized to a value of 1.0. The laser is then fired (by operating foot switch 121) and the detector 70 reads the received power at the delivery point and sends the information to the processor 114. Subtracter block 275 compares the received measurements to the selected reference 269a and generates an error signal. The error signal is provided to P1 controller 277 and the output of P1 controller 277 is added to the ACFAT 273a. Note that if the measured power is the same as the reference power, then the error signal is 0 and there is no adjustment to the ACFAT 273a. If the power at the output of SLA 30 is less than the reference power, a positive value that is proportional to the error and the dynamic response of the external control loop is added to the ACFAT. Conversely, if the measured power is greater than the reference, a negative value is added to the ACFAT 263a. After a number of iterations, the ACFAT 273a converges to a fixed value, thereby causing the reference signal and the measured power to approach equality.

According to some embodiments of the i-MP protocol, the power levels are proportional to the laser spot size and there is a need to ensure that the delivered power deviates by less than 5%. The power at the delivery end of SLA 30 is dependent on 3 major factors, namely the greatest linear dimension of the lesion, the weight of the patient and the skin pigmentation level of the patient. In order to minimize the error due to the non-linearity of the laser diode 103, three reference points are used and the auto-calibration device reads the actual delivered power at each of these 3 points:
lesion size smaller than 1.5 mm, use laser spot size of 1.5 mm;
lesion size between 1.5 mm and 3.0 mm, use laser spot size of 2.5 mm; and
lesion size greater than 3.0 mm, use laser spot size of 4.3 mm.

FIG. 3G is a flowchart of the method followed for the auto-calibration in some embodiments. In step 282 the type of optical delivery path to be calibrated is determined. This determination may be dependent on the output of the beam-width detector 158. In step 284, a desired laser power is obtained corresponding to the current optical delivery path. In step 286 the compensation factor ACFAT is initialized to 1.0 for the current optical delivery path. Then, in step 288 the laser controller 130 drives the laser to fire dependent on the desired laser power. In step 290, the detector 70 measures the power output at the end of the optical delivery path and provides the measured power to the processor 114. Then, in step 292 the subtraction block 175 compares the measured power and the desired power to generate an error signal. The PI controller 177 in step 294 adjusts the ACFAT so as to reduce the error signal. Step 296 checks whether the error signal is below a threshold value. If the error signal is still too high (the No option of step 296) then the control flow returns to step 290 to continue the auto-calibration. If the error signal is sufficiently small (the Yes option of step 296) then the ACFAT has been determined for the current optical delivery path and in step 298 the laser calibrator 132 checks whether there are more optical delivery paths to calibrate (i.e. whether the other spot sizes have yet been calibrated). If there are no more spot sizes then the calibration is complete (step 299). Otherwise, process flow returns to step 282 to perform the auto-calibration for the other delivery paths.

Each time the laser is fired, the auto-calibration device measures the output power detected by the photodiode and feeds it back into the laser console 10. The operational software then compares the power at the end of the delivery device with the power delivered by the laser cavity and feeds back the difference into the operational software through a Proportional Integral (P1) controller. The resulting digital power difference signal is used to calculate a new compensation factor for the slit lamp adaptor, which will compensate for all losses in the optical path from the laser cavity to the patient's eye. It will be understood that a Proportional Integral Derivative (PID) controller may also be used

As an example, if the slit lamp adaptor's determined transmission factor (FAT) is calculated at 75% for a particular spot size, the maximum output power at the patient's eye of a 2.5 W laser console will be 1875 mW and therefore, this will be the maximum power the user will be able to adjust the laser to when using the slit lamp adaptor as a delivery device for that particular spot size. On top of this gross transmission factor comes the determined compensation factor (ACFAT) from the auto calibration device, which can add up to 20% power compensation. If in the above example, an auto calibration routine is performed and the determined compensation factor is calculated at 10%, the maximum adjustable power will be 1687 mW. It will be appreciated by those skilled in the art, that the embodiment provides a laser system which can provide an improved accuracy in the desired power of the laser delivered to the patient's eye.

The auto-calibration procedure is described in more detail in co-pending application PCT/AU2006/000721 “A laser calibration method and system” with an international filing date of 29 May 2006, the contents of which are incorporated herein by cross-reference.

### Setting Parameters

In certain embodiments, referring now to FIGS. 4 to 6, there are shown flowchart diagrams of the steps involved in completing the setting of the necessary parameters required in the treatment procedure for the application device 10. The parameters include at least one dosage parameter, namely a quantity of ICG to be injected into the patient, and at least one application parameter such as the laser power to be used in the procedure. SET PARAMETER mode 300 guides the operator in entering the clinical parameters in order to determine the output power for the laser. The power setting of the laser is calculated by software running on processor 114 using the following equations:
P1=SZ*Mag*Kₗₐₛₑᵣ*K_{pig1}
P2=SZ*Mag*Kₗₐₛₑᵣ*K_{pig2}
where:
SZ=spot size selected at the SLA 30;
Mag=magnification of the retina laser lens 60 (typically 1.5);
Kₗₐₛₑᵣ=155.03875 W/mm² (Constant of Irradiance);
K_{pig1}=pigment factor 1;
K_{pig2}=pigment factor 2;
P1=output power in Watts for use in a first laser application; and
P2=output power in Watts for use in a second laser application.

Pigment factors 1 and 2 are based on an examination of the pigmentation of the patient's eye. In one arrangement the following values are used:

TABLE 1
Pigment factors

| Pigmentation | K_{pig1} | K_{pig2} |
|---|---|---|
| Low | 1.015 | 1.015 |
| Medium | 1.000 | 1.015 |
| High | 1.000 | 1.015 |

In some embodiments, when a “low” [hypo] pigmentation level is assessed, a higher power P1 is preferred to be used in the first laser application, as compared to the power P1 used in the first laser application for “medium” [normal] or “high” [hyper] assessed pigmentation levels. In particular, for example, the power P1 used in the first laser application for low assessed pigmentation can be about 0.5%, 1%, 1.5%, 2%, 2.5%, 3%, 3.5%, 4%, 4.5%, or 5% (e.g., about 0.25-5%, 0.5-4%, 0.5-3%, or 1-2%) higher than the power P1 used in the first laser applications for medium or high assessed pigmentation. In one preferred embodiment, the power P1 used in the first laser application for low assessed pigmentation is about 1-2% higher than the power P1 used in the first laser applications for medium or high assessed pigmentation. In some embodiments, in this regard, the same power P1 can be used for the first laser application for medium and for high assessed pigmentations.

In some embodiments, when a “high” pigmentation level is assessed, a lower power P1 is preferred to be used in the first laser application, as compared to the power P1 used in the first laser application for “medium” or “low” assessed pigmentation levels. In particular, for example, the power P1 used in the first laser application for high assessed pigmentation can be about 0.5%, 1%, 1.5%, 2%, 2.5%, 3%, 3.5%, 4%, 4.5%, or 5% (e.g., about 0.25-5%, 0.5-4%, 0.5-3%, or 1-2%) lower than the power P1 used in the first laser applications for medium or low assessed pigmentation. In one preferred embodiment, the power P1 used in the first laser application for high assessed pigmentation is about 1-2% lower than the power P1 used in the first laser applications for medium or low assessed pigmentation.

In some embodiments, the power P1 used in the first laser application is preferred to be lower than the power P2 used in the second laser application. This is applicable, in some embodiments, for low, medium, and high assessed pigmentation. In other embodiments, in the case of medium and high pigmentation, the power P2 used in the second laser application is higher than the power P1 used in the first laser application. In some embodiments, for example, the power P2 used in the second laser application is about 0.5%, 1%, 1.5%, 2%, 2.5%, 3%, 3.5%, 4%, 4.5%, or 5% (e.g., about 0.25-5%, 0.5-4%, 0.5-3%, or 1-2%) higher than the power P1 used in the first laser application. In one preferred embodiment, the power P2 used in the second laser application is about 1-2% higher than the power P1 used in the first laser application.

Additionally, although three levels of pigmentation are discussed above, it should be readily understood that additional level could also be used (e.g., 4 levels, 5 levels, 7 levels, or 2 levels). In some embodiments there will be one level of laser power (i.e., P1 and P2) to match each level of pigmentation. In other embodiments the level of P1 and P2 can be different at each pigmentation level or some may be the same.

The output power is recalculated when the first and the second laser applications are started or when a fundamental parameter is changed. The output power is calculated by software running on processor 114.

In some embodiments, one or more subsequent laser applications are performed to complete treatment. Such additional one or more laser applications may follow an assessment (e.g., via ICG angiography or other means) of the degree of completion of photo-activation of ICG or the presences of a complete photo bleaching (usually judged by the formation on the ICG angiography of an absolute dark spot at the irradiated area) in the targeted tissue and/or the presence of remaining non-activated ICG in the tissue. The one or more subsequent laser applications can comprise any suitable output power and duration. In one embodiment, for example, a single subsequent laser application (i.e., a third laser application) is administered at an output power that is similar or higher than the output power of the second laser application. Specifically, in this regard, the one or more subsequent laser applications (e.g., single third laser application) can have an output power that is about the same or 0.5%, 1%, 1.5%, 2%, 2.5%, 3%, 3.5%, 4%, 4.5%, 5%, 5.5%, or even 6% (e.g., about 0.5-5%, 1-4%, 1.5-3.5%, or 2-3%) higher than the output power of the second laser application. In some embodiments, it is preferred that a single subsequent laser application (i.e., a third laser application) has an output power that is about 2-3% higher than the output power of the second laser application. The duration of the third and subsequent laser application will typically be shorter than the first and second laser application. The third application will typically be about 15 to 60 seconds in duration (for example, about 15 to about 30 seconds, about 30 to about 45 seconds, about 45 to about 60 seconds, such as about 20, 25, 30, 35, 40, 45, 50 or 55 seconds).

To select SET PARAMETER mode 300 the operator presses the <MODE> button repeatedly until display 117 shows the message

<MODE> Parameter Adjustment

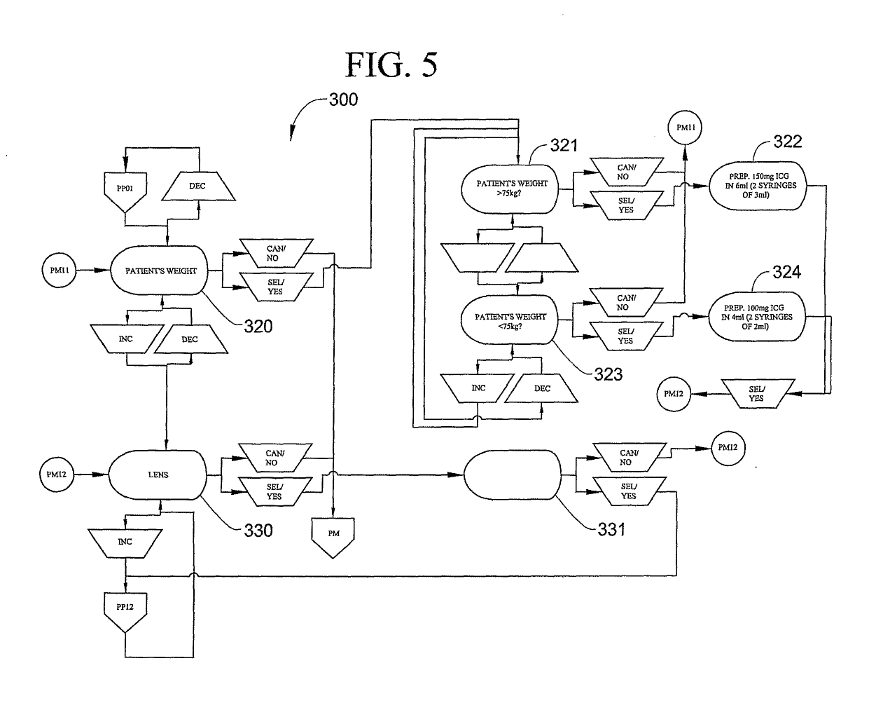

At this point the operator is presented with four selectable alternatives which include:
Lesion greatest linear dimension (GLD) 310 (see FIG. 4);
Patient's weight 320 (see FIG. 5);
Retina laser lens magnification 330 (see FIG. 5); and
Pigment type 340 (see FIG. 6).

The operator may step between these alternatives by pressing the INC and DEC buttons. The operator may then vary each of these options according to patient characteristics.

On selection of the lesion size mode 310 the operator is presented with three options to set the Lesion GLD. The choice between these options is based on an examination of the lesions in the patient's eye 100. The first of these options 311 corresponds to a lesion size less than 1.5 mm in which case the spot size (SZ) is to be set 312 to 1.5 mm. If this is appropriate as determined by examination of the patient, the operator is prompted 312 to turn the thumb wheel on SLA 30 to the appropriate spot size as indicated. The system will then return to mode selection level 200.

Alternatively, an operator can instruct the system to increase the lesion size parameter to be in the range 1.5 mm to 3.0 mm 313 in which case the spot size parameter stored in memory is set to 2.5 mm and the operator is prompted 314 to adjust the SLA 30 to a spot size of 2.5 mm. If the lesion size is indicated 315 as being greater than 3.0 mm, the spot size is set to 4.3 mm and a displayed message prompts 316 the operator to adjust the spot size at SLA 30.

Although the above description describes presenting the operator with three options for setting the lesion GLD, it should be readily understood by a person of skill in the art that any number of options can be presented to the user (e.g., 2, 3, 4, 5, 6, 7, 8, 9, or 10). Additionally, the spot size values can be varied (e.g., less than 1 mm, between 1 and 3 mm, between 1.6 mm and 6 mm, more than 1.6 mm, 3 mm, 4 mm, 1.75 mm, 2.5 nm, 3.5 mm, or 4.5 mm).

Keyboard 116 allows the operator to move between the various options for lesion size by using the <INC> or <DEC> buttons as appropriate. Once the operator has selected the appropriate lesion size and confirmed this by pressing the SEL/YES button, the appropriate prompt 312, 314, 316 is displayed on display 117.

Another parameter of importance is the patient's weight, as this will determine the amount of ICG (ICG) that will be required to be injected into the patient. When prompt 320 is displayed, the operator has the choice of entering the patient's weight. After selection of patient's weight mode by pressing the SEL/YES button, the operator may enter whether the patient's weight is either under 75 kg 321 or over 75 kg 323 by pressing the INC or DEC buttons. If the patient's weight is over 75 kg then the operator is instructed (by prompt 322 displayed on display 117) to prepare 150 mg of ICG in two syringes of 3 ml each. Alternatively if the patient's weight is less than 75 kg then the operator is instructed (by prompt 324) to prepare 100 mg of ICG in two syringes of 2 ml each. In addition to the above described option, in certain embodiments, additional weights may be presented to the operator for selection (e.g., above or below 65 kg, 70 kg, 80 kg, and 100 kg). Additionally, more than two values for patient weight may be presented (e.g., 3, 4, or 5).

Once this has been completed, the operator returns to the SET PARAMETER menu to confirm 330 the type of contact lens 60 that is to be used. In the illustrated embodiment only a single lens option is provided. Prompt 331 asks the operator to confirm that a Mainster Wide Field 1.5× magnification lens is used. In other embodiments, the operator may be presented with a potential choice of retinal laser lens types.

The final parameter to be selected in the SET PARAMETER mode is the pigment content of the eye that is to be treated. When prompt 340 is displayed on display 117, the operator is able to choose between a high pigment level 341, normal pigment level 342 and low pigment level 343 as determined by examination of the patient. The operator may step between prompts 341, 342 and 343 by pressing the INC and/or DEC buttons. When the appropriate pigment level is displayed on display 117, the operator selects the pigment level by pressing the SEL/YES button. Once this has been completed the process flow returns to the top level menu 200.

The parameters set in the SET PARAMETER mode are stored for use during the TREATMENT mode.

### Setting User Preferences

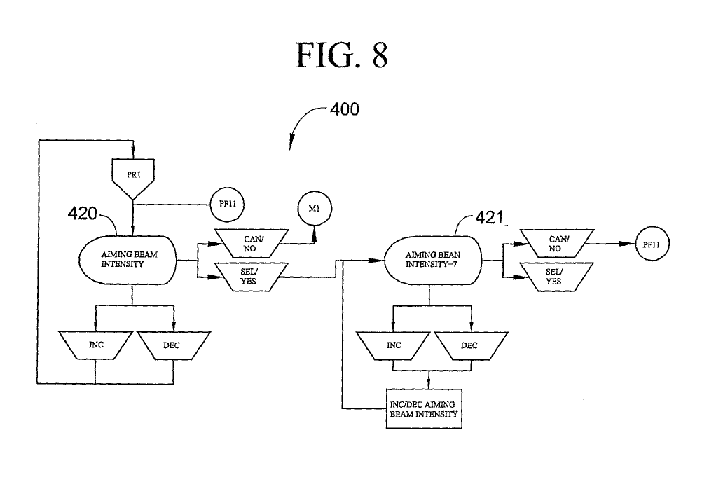

Referring now to FIGS. 7 and 8, there is shown a flowchart diagram of the steps involved in selecting features of the application device 10 which may be varied to suit the operator. In USER PREFERENCES mode 400, the operator can select some auxiliary parameters such as volume of the “beep” that is sounded by buzzer 115 and the intensity of the aiming laser beam emitted by laser diode 113.

To select USER PREFERENCES mode 400 the <MODE> button is pressed repeatedly until display 117 shows the message

<MODE> User Preferences

If the operator presses the MODE button again, the display changes to the next mode (TREATMENT MODE). Alternatively, if the operator enters the USER PREFERENCES mode by pressing the SEL/YES button, the operator is presented with a set of parameters to vary. The operator may step between these parameters by pressing the INC and/or DEC buttons on keyboard 116. Prompt 410 enables the operator to adjust the sound intensity level. By pressing the INC or DEC buttons, the sound intensity may be adjusted to a number ranging between 4 and 9. Displayed message 411 indicates the current setting of the sound intensity.

Similarly, as seen in FIG. 8, prompts 420 and 421 allow the operator to adjust the aiming beam intensity in a range of 2 to 9. As described above, the aiming beam is a visible laser of relatively low power output that is used to position and aim the laser subsequently used in the therapeutic procedure.

### Treatment Mode

In certain embodiments, once the various treatment parameters and user preferences have been adjusted according to the specific requirements of both the patient and the operator, the treatment protocol may be commenced.

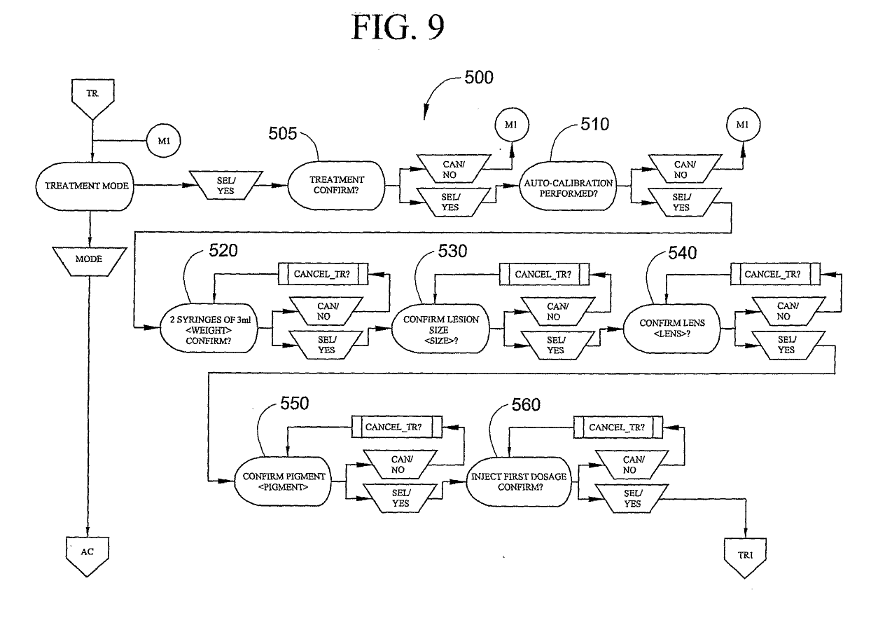

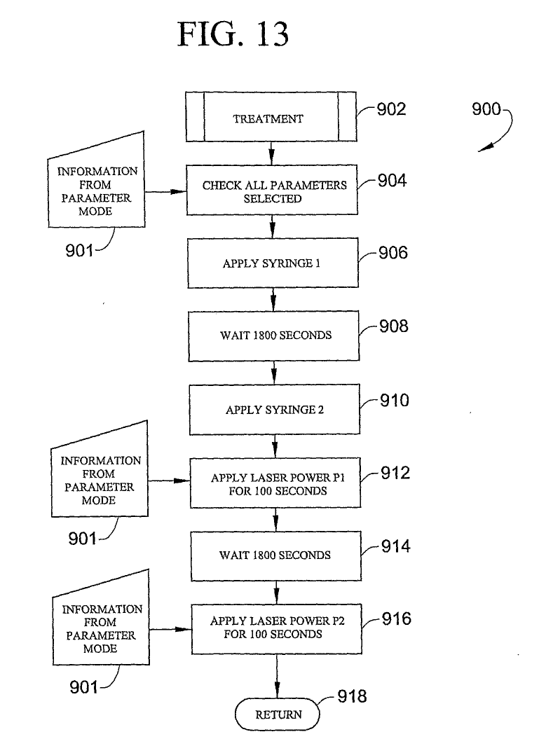

FIG. 13 provides an overview 900 of the treatment protocol. More detail is shown in FIGS. 9 to 12. The treatment protocol has a timing sequence. The control software described herein guides the operator in executing the treatment according to the timing sequence.

The procedure commences at 902. In step 904, information 901 saved during the SET PARAMETER mode is displayed on display 117 in order for the operator to check that the parameters have been selected appropriately. Next, in step 906, the operator injects a first syringe of ICG into the patient. Amer waiting 1800 seconds (step 908), the operator injects a second syringe of ICG into the patient (step 910). Software running on processor 114 guides the operator through steps 906-908 by providing prompts and instructions at appropriate times during the procedure.

After both syringes of ICG have been injected, in step 912 laser power P1 is applied to a treatment area in the patient's eye for 100 seconds. The operator applies the laser power by pressing foot pedal 121. The power setpoint of the laser is determined by software running on microprocessor 114 dependent on the information 901 entered in the SET PARAMETER mode.

After the first laser application there is a further wait of 1800 seconds (step 914). The control system 90 times the wait and alerts the operator near the end of the wait. Then, in step 916, laser power P2 is applied to the treatment area for 100 seconds. Information 901 entered in the SET PARAMETER mode is used to calculate the required laser power P2.

The procedure is then complete and the controlling software returns (step 918) to higher-level selection menus.

Referring now to FIGS. 9 to 12, there are shown flowchart diagrams of the steps 500 involved in guiding the operator through the ICG mediated photothrombosis procedure employing as application device the laser unit 10.

Once the operator has confirmed (by pressing the SEL/YES button in response to prompt 505) that a treatment run is to commence, the system initially prompts the operator to confirm that a number of important actions have been performed before the treatment can commence. In this manner, the system makes use of the various parameters that have already been entered into the system to ensure that the correct treatment protocol is being followed.

To select TREATMENT mode the <MODE> button is pressed repeatedly until display 117 shows the message

<MODE> Treatment

Alternatively, TREATMENT mode 500 may be activated directly by pressing the <Treat> button on keyboard 116.

In TREATMENT mode 500, the operator is first asked (by means of prompt 510 displayed on display 117) to confirm that an auto-calibration has been recently performed. In one arrangement the system requires that an auto-calibration is performed at least once every 10 treatments. Next the system confirms the weight of the patient and the amount of ICG that is to be delivered. For example, prompt 520 indicates that 2 syringes of 3 ml each are to be used, and asks the operator to confirm this. The operator responds by pressing the CAN/NO button or the SEL/YES button, as appropriate.

Following this, the selected lesion size is displayed 530 and the operator is prompted to confirm that the lesion size agrees with the spot size selected on SLA 30. The system then prompts 540 the operator to confirm which contact lens 60 is used. In the described example, a Mainster WF lens is used as the contact lens 60. The system also prompts 550 the operator to confirm that the pigment concentration parameter is correctly set. Prompts 510, 520, 530, 540, 550 are part of a final safety check to ensure that the system is parameterized correctly with regard to the patient to be treated.

The system next prompts 560 the operator to inject the first dose of ICG into the patient's blood circulation. After the first syringe has been injected, the operator confirms the injection by pressing the SEL/YES button. Alternatively, the operator may halt the procedure by pressing the CAN/NO button. If the injection is confirmed, software running on processor 114 sets a count down timer to 1680 seconds. The current value of the timer is displayed 610 on display 117. The operator may cancel the timer by pressing the CAN/NO button.

When the timer has finished the count-down, the controlling software resets the timer to 120 seconds and causes the buzzer 115 to sound a continuous beep (step 615). The system continues counting down and prompts 620 the operator to put on safety glasses and confirm that this has been done. If the operator presses the SEL/YES button to confirm use of the safety glasses, processor 114 switches off the buzzer 115 and switches on the aiming laser diode 113. A prompt 640 is then displayed instructing the operator to place lens 60 on the patient's eye and confirm that this has been done. Prompt 650 then instructs the operator to position the aiming laser beam on the lesion and to confirm that this action has been performed. The display 117 continues to display 660 the status of timer T1.

After the timer has completed the 120 second countdown (during which time the operator has put on safety glasses, positioned lens 60 and aimed the laser), the timer is reset to 60 seconds (step 675). Prompt 670 is displayed instructing the operator to inject the second syringe into the patient. The operator is required to confirm, by pressing the SEL/YES button, that the second syringe has been injected into the patient. If this confirmation has not occurred within 60 seconds the treatment will automatically time out and display 117 presents the message Treatment time-out 676.

The system then, in step 685, starts a 90 second countdown timer. Screen display 680 displays the timer and also the calculated power parameter P, the lens magnification and the spot size. In the example shown, P=375 mW, L=1.5 and the spot size is 1.5 mm. When the timer reaches the last 30 seconds of the countdown period, 3 long beeps are sounded. When the timer reaches the last 20 seconds, 2 long beeps are sounded and at the last 10 seconds one long beep will be emitted. At the last 5 seconds one beep is sounded at each second. At the end of the 90 second period, the system displays a prompt 710 instructing the user to press the foot pedal 121. If the operator does not press the foot pedal within 60 seconds (set in step 705), the procedure times out and an error signal is displayed. The operator also has the option of canceling the procedure by pressing the CAN/NO button.

If the operator presses the foot pedal 121, a sound signal is emitted by buzzer 115 and the main laser diode 103 is activated (step 720). At this stage, the patient is positioned and the lens is in place. A 100 second countdown timer is started in step 725. During the application of the main laser, the power, lens magnification and spot size are displayed 730 and an indicator of delivered energy dosage is also displayed. The display 730 also includes an indication that the first laser application is underway.

The foot pedal 121 must be kept pressed. If the foot pedal 121 is released the laser diode 103 and countdown are paused (step 735). The countdown and laser treatment can be resumed (step 736) by pressing foot pedal 121 again. In the event of foot pedal 121 not being pressed for more than 30 seconds, the system will display the time-out message. The operator may also cancel the procedure by pressing the CAN/NO button.

At the end of the 100 second period, the controlling software running on processor 114 deactivates the treatment laser diode 103 and aiming laser diode 113 (step 740). A 1680 second countdown timer is then started in step 745. The delivered energy (DE) dose indicator remains on display 750 whilst the laser assembly 80 is in standby mode. In the example shown the delivered energy is 4205 J.

When the 1680 second countdown is complete, the timer is reset to 120 seconds in step 765, providing an overall delay of 1800 seconds. The buzzer 115 sounds a continuous beep 760 to alert the operator. If the operator presses the SEL/YES button, the processor 114 acts (in step 770) to turn off the beep and turn on the aiming laser diode 113. The operator is then prompted 775 again to place the lens 60 on the eye 100 to be treated and to confirm that the lens 60 is in place. The software displays prompt 780 to instruct the operator once again to position the aiming laser on the treatment area including the lesion. If the operator confirms that this has been done (by pressing the SEL/YES button), then prompt 790 is displayed, prompting the operator to prepare for the second laser application. If the operator presses the SEL/YES button or the countdown timer reaches the end of the 120 seconds, the tinier is reset to 60 seconds in step 816 and prompt 810 is displayed instructing the operator to press the foot pedal. The settings of power P, lens magnification and spot size are also displayed.

If the operator does not press the foot pedal within the 60 second window, then in step 815 the buzzer 115 sounds an alert.

Once the foot pedal 121 is pressed the main laser diode 103 is activated in step 820 and a countdown period of 100 seconds commences (step 830). An indicator of energy dosage is displayed 840 along with power, lens magnification and spot size and an indication that this is the second application of the laser assembly 80. As described above, the power P2 used in the second laser application may be higher than the power P1 used in the previous laser application. The relative strength of P1 and P2 is determined by the pigmentation parameter K_{pig}.

The foot pedal 121 must be kept pressed down. If the foot pedal 121 is released the laser and countdown are paused (step 845). These can be resumed (step 846) by pressing the foot pedal 121 again. If the foot pedal 121 is not pressed for more than 30 seconds, application device 10 will sound a continuous beep until the foot pedal 121 is pressed again.

At the end of the 100 second period, the treatment laser diode 103 and aiming laser diode 113 are deactivated in step 847. The delivered energy dose indicator remains on the display 117 (step 850) and the operator is given the option to return to the top level menu display 200. At various stages throughout the procedure the treatment may be cancelled explicitly by the operator or alternatively if a time out period has been exceeded.

Exemplary i-MP Protocol

The following description is an exemplary embodiment of a Protocol for the Evaluation of the Indocyanine Green-mediated Photothrombosis (i-MP), in the Treatment of the Occult Subfoveal Neovascular Membranes of the Age-Related Macular Degeneration (AMD).

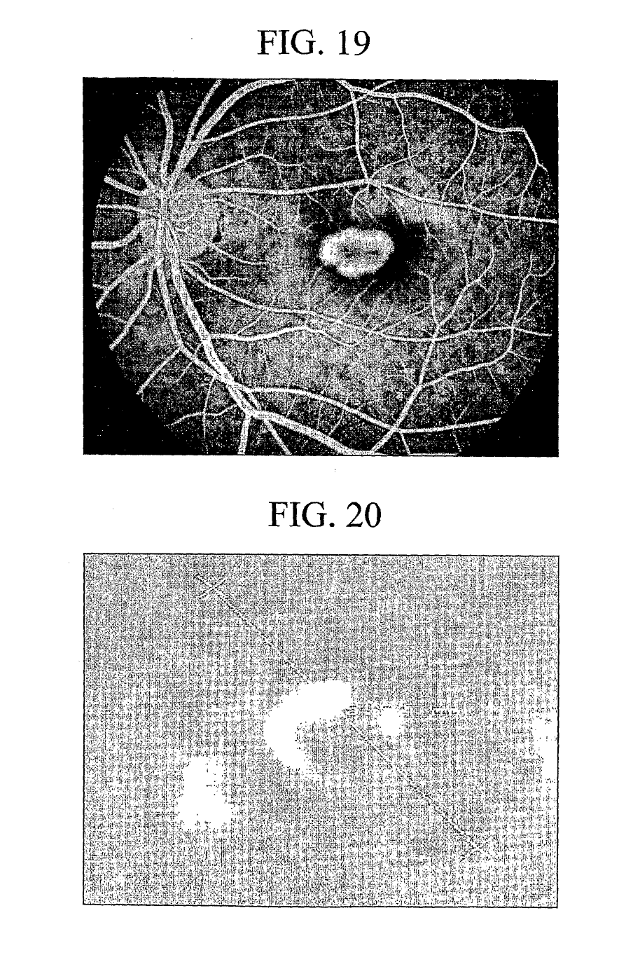

The inclusion criteria may be:
Age, 50 years or more;
Visual Acuity (VA) with correction between 20/40 and 20/400 for farsighted, according to Snellen's Table (34-73 letters);
Exudative macular degeneration with predominantly occult subfoveal membrane, with the highest linear dimension, less than, or equal to 3,500 μm;
Presence of classic CNV (e.g., as shown in FIGS. 19 and 20);
Express consent signed by the patient and/or his/her legal proxy.
Observation: Occult membranes are considered as being the fibrovascular displacements of the retinal pigment epithelium. (RPE) and the late vessel leakage which origins could not be verified by the fluoresceinography.

The Exclusion Criteria may include:
Previous retinal surgery or macular photocoagulation in his/her medical history;
Opacity of the ocular means that could ruffle the observation of the macular region and/or the interpretation of the fluorescein angiography;
Presence of other ocular diseases that could diminish the central visual acuity (for instance: diabetic retinopathy, venous thrombosis, optical neuropathies, other maculopathies, etc);
Allergy to fluorescein;
Degenerative myopia;
Use of anticoagulant and platelet anti-adhesive.

The exemplary methodology may include:
Thorough opthalmologic examination: visual acuity assessment and bio-macroscopic evaluation of the maculae.
Colour retinography and fluorescein angiography (5 ml of 10% or 2.5 ml of 20% fluorescein) with the initial photographs taken 10 seconds after the injection and the last photographs after 10 minutes.
Photo sensitive drug: Indocyanine Green (ICG)

The ICG dose may be calculated as follows:
Maximum dose allowed: 2 mg/kg
Indocyanine Green (50 mg in 2 ml)
Patients:
   Up to 50 kg: 100 mg of ICG
   50-75 kg: 125 mg of ICG
   more than 75 kg: 150 mg of ICG

The solution may be prepared as follows:
Total volume: 4, 5, 6 ml
2 vials (3 or 5 ml)
   2 ml=50 mg
   2.5 ml=62.5 mg
   3 ml=75 mg
Put 2 ml of distilled water in a flask of 50 mg of ICG (1 ml=25 mg)
Shake the solution for 1 (one) minute
Prepare the necessary amount (100, 125, 150 mg)
Keep the solution protected from light

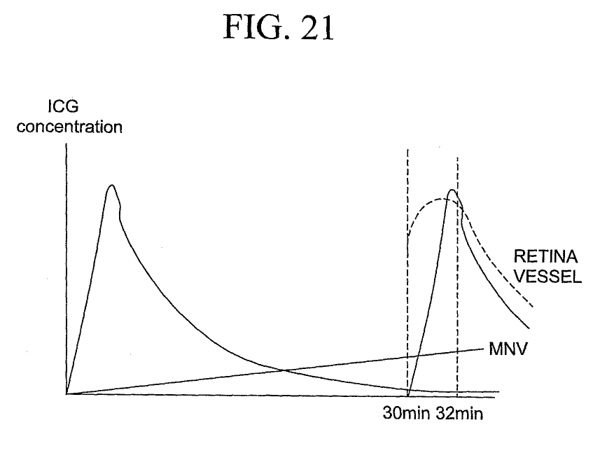

The infusion may be prepared as follows:
Separate the mixture in 2 (two) equal parts (50 mg, 62.5 mg, 75 mg):
First infusion:
   Angiographic study
   Loading and initial sensitiveness
Second infusion:
   Maximal final impregnation
   Time: 30 minutes after the first infusion
   Observation: 2 minutes after the second infusion it has the maximal concentration in the membrane. Therefore after 32 minutes we can start the laser treatment (see, for example FIG. 21)

Infusion may be as follows:
Venous Access with “abocath”
Adjust the chronometer to 30 minutes
First infusion followed or not by a flush (for angiography)

Time of the infusion may be as follows:
1-2 minutes after the second infusion, it is when we will have the maximal concentration of contrast in the membrane. Therefore after 32 minutes we do the laser.

Laser Aim Beam Spot Size may be as follows:
Cover the lesion area with 500-1000 μm of safety boundaries
Consider the variables:
   Size
   Delineation
   Lens/Aim Beam combination

Final preparation may be as follows:
Goldman Lens
   Image Magnification (IM): 1.0×
   Laser Spot Size Magnification (LSM): 1.0×
Mainster Wilde Field Lens
   Image Magnification (IM): 0.7×
   Laser Spot Size Magnification (LSM): 1.5×
PDT Lens
   Image Magnification (IM): 0.6×
   Laser Spot Size Magnification (LSM): 1.6×

In certain specifications of the lens, the above parameters may be written in a manner such that Image Magnification=Lens Magnification and Spot Magnification=Laser Spot Size Magnification

Lasertherapy parameters may be as follows:
1 or 2 laser applications
Exposure time: 100 seconds
Power:
   Spot size at the retina=2.25→Power=330-350 mW
   Spot size at the retina=3.75→Power=560-590 mW
   Spot size at the retina=6.45→Power=970-990 mW
If the spot in the retina is 8.00 mm for example, make a rule of 3 (three) to calculate the power. In this case, we will have: Power=8.50×990/6.45=1.304 mW. After this, reduce the power in 20%, that means, use the power of 1.040 mW for the treatment.
Use the Opto TripleT™ Laser Photocoagulator.

Parameters Selection for the Lasertherapy on the Opto TripleT™ Laser Photocoagulator may be as follows:
1. Aim Beam Intensity
   Press the PARAMETER button of your equipment;
   Adjust the aim beam intensity with the INCREMENT and DECREASE buttons;
   Aim Beam Intensity: 8
2. Image Magnification Adjustments:
   Press the PARAMETER button of your equipment;
   Adjust the aim beam intensity with the INCREMENT and DECREASE buttons. Keep the button pressed for faster adjustment
   Image Mag.: 0.60
3. Laser Spot Magnification Adjustment:
   Press the PARAMETER button of your equipment;
   Adjust the aim beam intensity with the INCREMENT and DECREASE button. Keep the button pressed for faster adjustment.
   Laser Spot Mag: 1.66
   The Image Magnification and the Laser Spot Magnification parameters, are both related to the lens used (one is the opposite of the other), therefore when one is modified, the other is modified simultaneously.

The Main Screens of the Lasertherapy with the Opto TripleT™ Laser may include the following information:
Standby Status
1. First Line:
   Irradiance in W/cm² (I),
   Image Magnification (L)

Laser Spot diameter in mm;
2. Second Line:
   Power in mW
   Duration in seconds (s)
   Total Energy Density in J/cm².
   Treatment Status
1. First Line:
   Irradiance in W/cm² (I),
   Remaining time in seconds (s)

Applied Energy Density in J/cm².
2. Second Line:
   Power in mW,
   Duration in seconds (s)

Total Energy Density in J/cm².

The final preparations may include the following steps:
Position the lens;
Start the second infusion followed by the flush with 5 ml of 0.9% SF;
Wait for 2 (two) minutes;
Begin the laser application

The first light application may include the following steps:
If any alteration is detected during the application, lower the power 10 to 20 mW and continue the Lasertherapy;
If there is any detectable secondary edema after the first light application, the power can be increased from 10 to 20 mW;
End of treatment.

Care after the treatment may include:
Avoid any intense physical effort for one week;
Avoid direct exposure to sunlight.

Follow-up may include:
Clinical Opthalmologic evaluation: 7 days;
Evaluation after 30 days: sodic fluorescein and green Indocianine angiography;
Angiography and OCT every 3 months;
The occult lesion treatment, must not exceed more than 50% over the classic one.

### EXAMPLES

### Example 1 (Prophetic)

### Comparison of Intravitreal Ranibizumab Treatment Alone with Combined Intravitreal Ranibizumab/ICG-Mediated Photothrombosis Treatment

The synergistic effects of combining intravitreal ranibizumab treatment with i-MP treatment in patients with AMD may be determined using a multi-centre, parallel group, open labeled, randomized, non-inferiority trial of the safety and efficacy of intravitreal ranibizumab treatment (standard regimen; monthly injections) alone in comparison with combined treatment using i-MP and intravitreal ranibizumab for the management of choroidal neovascularization in age-related macular degeneration. A non-inferiority trial is appropriate in this case to show that the treatment will have at least 90% of the same efficacy as the standard treatment, while achieving other advantages such as greater availability, increased patient compliance, reduced cost, less invasiveness, reduced side effects, and/or reduced dosage or frequency of administration.

The study includes three treatment arms: the first study arm will receive the standard ranibizumab regimen of 0.5 mg monthly intravitreal injections. The second study arm will receive a customized regimen of 0.5 mg of intravitreal ranizumab injection every 12 weeks with one session of i-MP performed within ±3 weeks of the injection. The third study arm will receive a customized regimen of 0.5 mg of intravitreal ranizumab injection every 18 weeks with one session of i-MP performed within ±3 weeks of the injection.

In order to obtain 82% statistical power with a 1-sided alpha equal to 0.025%, the study will have 245 patients based on the following assumptions:

A) the standard therapy has a 95% chance of preventing moderate visual acuity loss (<15 letters as measured using the Early Treatment Diabetic Retinopathy Study (ETDRS) visual acuity scale) for the reference therapy (ranibizumab);

B) the combination therapies need at least an 85% chance of preventing moderate visual acuity loss (<15 ETDRS letters) (A=10%); and

C) a 10% rate of dropout from the trial.

In order to adequately assess the non-inferiority of the combined therapies, a minimum of 74 patients completing the study per treatment group is needed.

In addition to visual acuity, the effect on retinal thickness over time may be assessed using optical coherence tomography measurements and the change in total lesion area/area of CNV may be assessed over time using fluorescein angiography and compared. It is expected that at least 85% of the patients in the second and/or third study arms will at least maintain the same visual acuity as measured by ETDRS letters and that at least 50% of the patients receiving these treatments will have improved visual acuity as measured by ETDRS letters. These results are expected to be at least a 10% improvement over the results from the first study arm and will represent a reduction in frequency of dosing at about 67%.

### Example 2 (Prophetic)

### Comparison of Intravitreal Bevacizumab Treatment Alone with Combined Intravitreal Bevacizumab/ICG-Mediated Photothrombosis Treatment

The synergistic effects of combining intravitreal bevacizumab treatment with i-MP treatment in AMD patients may be determined using a multi-centre, parallel group, open labeled, randomized, non-inferiority trial of the safety and efficacy of intravitreal bevacizumab treatment (standard regimen; injections every 6 weeks) alone in comparison with combined treatment using i-MP and intravitreal bevacizumab for the management of choroidal neovascularization in age-related macular degeneration. A non-inferiority trial is appropriate in this case to show that the treatment will have at least 90% of the same efficacy as the standard treatment, while achieving other advantages such as greater availability, increased patient compliance, reduced cost, less invasiveness, reduced side effects, and/or reduced dosage or frequency of administration.

The study includes three treatment arms: the first study arm will receive the standard bevacizumab regimen of one 1.5 mg intravitreal injection every 6 weeks. The second study arm will receive a customized regimen of one 1.5 mg of intravitreal bevacizumab injection every 12 weeks with one session of i-MP performed within f 3 weeks of the injection. The third study arm will receive a customized regimen of one 1.5 mg of intravitreal bevacizumab injection every 18 weeks with one session of i-MP performed within ±3 weeks of the injection.

In order to obtain 82% statistical power with a 1-sided alpha equal to 0.025%, the study will have 245 patients based on the following assumptions:

A) the standard therapy has a 95% chance of preventing moderate visual acuity loss (<15 letters as measured using the Early Treatment Diabetic Retinopathy Study (ETDRS) visual acuity scale) for the reference therapy (ranibizumab);

B) the study therapy has an 85% chance of preventing moderate visual acuity loss (<15 ETDRS letters) (Δ=10%); and

C) a 10% rate of dropout from the trial.

In order to adequately assess the non-inferiority, of the combined therapies, a minimum of 74 patients per treatment group is needed.

In addition to visual acuity, the effect on retinal thickness over time may be assessed using optical coherence tomography measurements and the change in total lesion area/area of CNV may be assessed over time using fluorescein angiography and compared. It is expected that at least 85% of the patients in the second and/or third study arms will at least maintain the same visual acuity as measured by ETDRS letters and that at least 50% of the patients receiving these treatments will have improved visual acuity as measured by ETDRS letters. These results are expected to be at least a 10% improvement over the results from the first study arm and will represent a reduction in frequency of dosing at least 50%.

### Example 3 (Prophetic)

### Comparison of Intravitreal Pegaptanib Treatment Alone with Combined Intravitreal Pegaptanib/ICG-Mediated Photothrombosis Treatment

The synergistic effects of combining intravitreal pegaptanib treatment with i-MP treatment in AND patients may be determined using a multi-centre, parallel group, open labeled, randomized, non-inferiority trial of the safety and efficacy of intravitreal pegaptanib treatment (standard regimen; injections every 6 weeks) alone in comparison with combined treatment using i-MP and intravitreal pegaptanib for the management of choroidal neovascularization in age-related macular degeneration. A non-inferiority trial is appropriate in this case to show that the treatment will have at least 90% of the same efficacy as the standard treatment, while achieving other advantages such as greater availability, increased patient compliance, reduced cost, less invasiveness, reduced side effects, and/or reduced dosage or frequency of administration.

The study includes three treatment arms: the first study arm will receive the standard pegaptanib regimen of one 0.3 mg intravitreal injection every 6 weeks. The second study arm will receive a customized regimen of one 0.3 mg of intravitreal pegaptanib injection every 12 weeks with one session of i-MP performed within ±3 weeks of the injection. The third study arm will receive a customized regimen of one 0.3 mg of intravitreal pegaptanib injection every 18 weeks with one session of i-MP performed within ±3 weeks of the injection.

In order to obtain 82% statistical power with a 1-sided alpha equal to 0.025%, the study will have 245 patients based on the following assumptions:

A) the standard therapy has a 70% chance of preventing moderate visual acuity loss (<15 letters as measured using the Early Treatment Diabetic Retinopathy Study (ETDRS) visual acuity scale) for the reference therapy (ranibizumab);

B) the study therapy has an 67.5% chance of preventing moderate visual acuity loss (<15 ETDRS letters) (Δ=2.5%); and

C) a 10% rate of dropout from the trial.

In order to adequately assess the non-inferiority, of the combined therapies, a minimum of 74 patients per treatment group is needed.

In addition to visual acuity, the effect on retinal thickness over time may be assessed using optical coherence tomography measurements and the change in total lesion area/area of CNV may be assessed over time using fluorescein angiography and compared. It is expected that at least 50% of the patients in the second and/or third study arms will at least maintain the same visual acuity as measured by ETDRS letters and that at least 25% of the patients receiving these treatments will have improved visual acuity as measured by ETDRS letters. These results are expected to be at least a 20% improvement over the results from the first study arm and will represent a reduction in frequency of dosing at least 50%.

### Example 4

### Indocyanine Green Mediated Photothrombosis with and without Bevacizumab Intravitreal for Treatment of Choroidal Neovascularization Subfoveal Secondary to AMD

Purpose: To determine efficiency, safety, and clinic response obtained with indocyanine green mediated photothrombosis (i-MP) with and without intravitreal Bevacizumab (Avastin), in patients with subfoveal choroidal neovascularization (CNV) secondary to age-related macular degeneration (AMD), in accordance with certain embodiments.

Method: In this study an infrared diode laser (i-MP), supplied by Opto Global Holdings Pty Ltd, Adelaide, Australia, was used combined with the application of intravitreal Bevacizumab.

Fifty patients (50 eyes) where studied in the age group of between 50 and 70 years, split into two groups. Group A was treated with indocyanine green mediated photothrombosis (i-MP) and the application of intravitreal Bevacizumab and Group B was treated with indocyanine green mediated photothrombosis (i-MP) only. Twenty-five eyes were treated with i-MP combined with an intravitreal injection of 2 mg (0.08 ml) of Bevacizumab administered 15 days after the laser application (Group A), and twenty-five eyes were treated with i-MP only (Group B). The Patients in both groups had a follow-up with fluorescein angiography and optical coherence tomography (OCT) after about 10 months. In some patients it was necessary to perform two treatment sessions with i-MP.

The technique consists of two stages: the infusion of indocyanine green intravenously and the laser application. The infusion stage of indocyanine green, consists of two applications, both of 50 mg and separated by 20 minutes. The laser application stage, also consisted of two sessions, the first application is delivered two minutes after the second dose of green indocyanine and the second laser application was delivered twenty minutes after the first application.

The patients were recommended to abstain of solid foods and dairy products for two hours prior to the treatment, in the case there are very fine or difficult to observe veins. It was recommended that the patients drink abundant water two hours before the treatment and to discontinue the anticoagulants and ginkgo biloba seven days before the treatment.

To accomplish the dissolution of the indocyanine green, for each infusion, we use 2 ml of distilled water in a flask of 50 mg of indocyanine green (1 ml=25 mg), we agitate the solution during 1 minute and protect the solution from light. For the first infusion, for the venous access, we use an abocath and we adjust the chronometer for 30 minutes. We inject the first dose (2 ml) and after thirty minutes, we inject the second dose (2 ml).

The parameters of the laser used are: exposition 100 seconds, power dependent on the spot size in the retina and the size of the CNV (including the magnification of the laser lens in use), normally we use a spot of 2.5 mm with a Mainster lens.

Care was taken with this treatment, if there was a retina coloration change, then we decreased the power 10-20 mw. If alteration of the coloration was observed in the first application, then we discontinued the second application. If there was an alteration of the coloration in the second application, then we applied the other laser irradiation for 20 minutes without infusing indocyanine green.

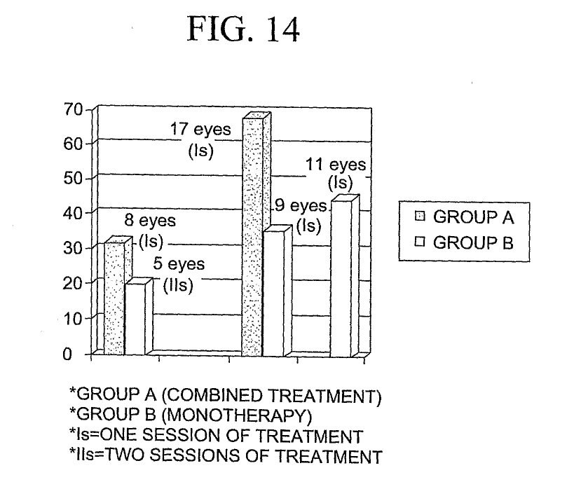

Results: FIG. 14 shows the clinical findings of 25 eyes treated with the combination of i-MP+intravitreal Bevacizumab (Group A) and 25 eyes with i-MP as monotherapy (Group B). Table 1 (below) shows characteristics of the choroidal neovascular membranes, of 25 eyes treated with the combination of i-MP+intravitreal Bevacizumab (Group A) and i-MP as monotherapy (Group B). Group A, during 10 months of follow-up, showed stabilization in the visual acuity in 8 eyes with staining in the fluorescein angiography (32%), in 17 eyes there was an improvement of the visual acuity in 2 lines also with a staining at level of the fluorescein angiography (68%). In Group A, it was not required to perform a second treatment session. In Group B, after 6 months of follow-up, it was observed a reduction in the visual acuity in 5 eyes with leakage of 40% in the choroidal neovascular membranes as evidenced by fluorescein angiography. In 10 months of follow-up, 9 eyes presented stability of the visual acuity with staining in the fluorescein angiography (36%) and in 11 eyes there was an improvement of the visual acuity of almost 2 lines with staining in the fluorescein angiography (44%). In Group B, it was necessary to perform a second treatment session in 5 eyes in 6 months of follow-up (20%). Both groups were evaluated with fluorescein angiography and OCT.

TABLE 1

| | Predominantly Classic | Minimumly Classic | |
|---|---|---|---|
| Groups | (>50%) | (<50%) | Occult |
| A | 52.00 | 25.00 | 23.00 |
| B | 48.00 | 33.70 | 18.30 |

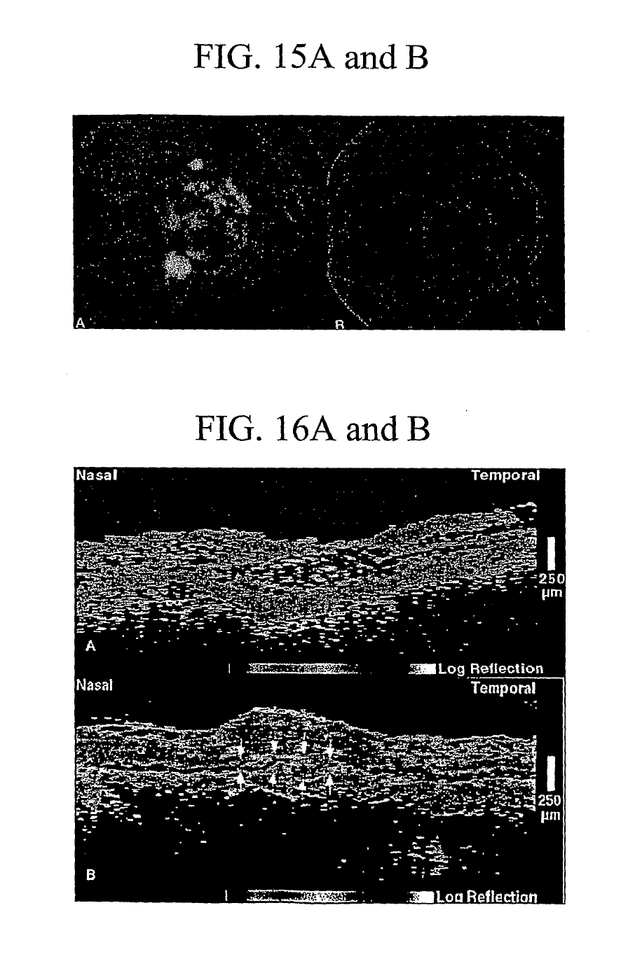

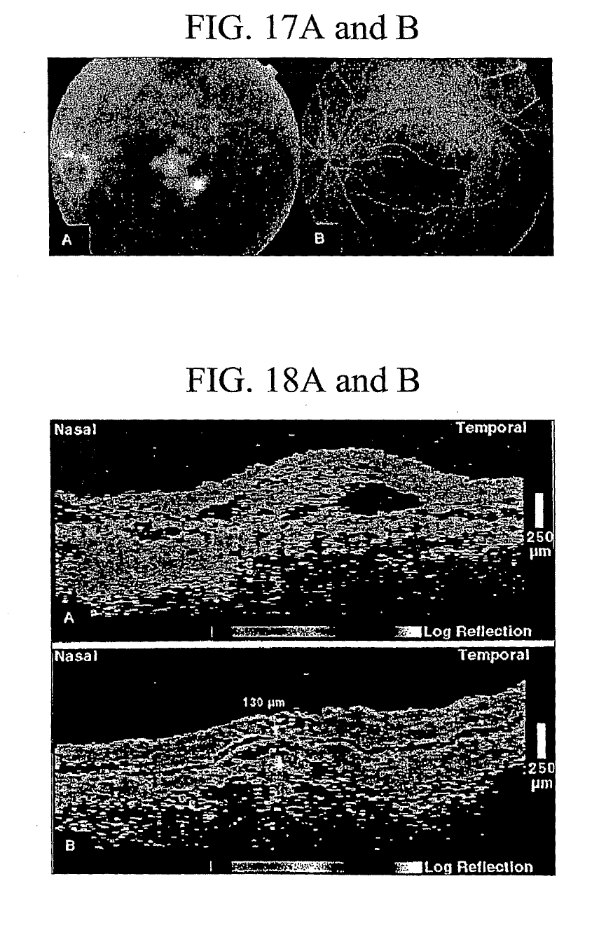

FIGS. 15A and 15B show the angiography fluorescein of a male patient, of 65 years old, with a visual acuity in right eye of count fingers at 2 mts, presenting a classic CNV. The treatment was i-MP as a monotherapy. FIG. 15A shows the image obtained prior to treatment and FIG. 15B shows the image obtained about 10 months after the treatment. FIGS. 16A and B illustrate the OCT of a male patient, of 65 years old, with a visual acuity in right eye of count fingers at 2 mts, presenting a classic CNV. FIG. 16A shows the image obtained prior to treatment and FIG. 16B shows the image obtained 10 months after the treatment. FIGS. 17 and 18 show the Fluorescein Angiography (FIGS. 17A and B) and OCT (FIGS. 18A and B) of a patient, with a visual acuity in the left eye of 0.1, presenting a occult CNV. FIGS. 17 and 18 show the results from the combined treatment, i-MP+intravitreal Bevacizumab before treatment and 10 months after treatment. The patient obtained a visual acuity of 0.3. Choroidal neovascular membrane with leakage for extravasation of contrast by detachment of the retinal pigment epithelium, (RPE) and in OCT presents increase in the reflectivity from the RPE and elevation. FIGS. 17A and 18A show the images obtained prior to treatment. FIGS. 17B and 18B show the image obtained 10 months after treatment. The angiography shows hipofluorescence by scar, without activity of the membrane in the OCT.

Conclusions: The choroidal neovascular membranes, responded better to the combined treatment (indocyanine green mediated photothrombosis followed by intravitreal injection of Bevacizumab fifteen days after the laser treatment), presenting cicatrization of the CNV in 10 months of follow-up, evaluated by fluorescein angiography and OCT.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating CNV secondary to wet AMD, Angioid Streaks, Pathologic Myopia, Central Serous Retinopathy, or other choroidal diseases resulting from inflammatory conditions and idiopathic causes comprising:
administering a medicament in combination with i-MP treatment, wherein the medicament is not triamcinolone or triamcinolone acetonide.

2. A method of treating CNV secondary to wet AMD, Angioid Streaks, Pathologic Myopia, Central Serous Retinopathy, or other choroidal diseases resulting from inflammatory conditions and idiopathic causes comprising:
administering an antiangiogenesis compound in combination with i-MP treatment.

3. The method according to claim 2, wherein the antiangiogenesis compound comprises an anti-VEGF compound.

4. The method of claim 3, wherein the anti-VEGF compound comprises an antibody or antibody fragment.

5. The method of claim 3, wherein the antibody or antibody fragment comprises bevacizumab.

6. The method of claim 3, wherein the antibody or antibody fragment comprises ranibizumab.

7. The method of claim 3, wherein the anti-VEGF compound comprises an aptamer.

8. The method of claim 7, wherein the aptamer comprises pegaptanib sodium.

9. The method of claim 3, wherein the antiangiogenesis compound decreases extracellular protease expression and/or inhibits endothelial cell migration.

10. The method of claim 9, wherein the antiangiogenesis compound comprises a steroid-based compound.

11. The method of claim 10, wherein the steroid-based compound comprises anecortave acetate.

12. The method of claim 3, wherein the antiangiogenesis compound is vatalanib.

13. The method of claim 1, wherein the medicament or antiangiogenesis compound is administered up to three weeks after i-MP treatment.

14. The method of claim 13, wherein the medicament is administered immediately after i-MP treatment.

15. The method of claim 13, wherein the medicament is administered up to 1 week after i-MP treatment.

16. The method of claim 13, wherein the medicament is administered up to 2 weeks after i-MP treatment.

17. The method of claim 13, wherein the frequency of administration of the medicament or antiangiogenesis compound in combination with i-MP treatment required for therapeutic efficacy reduces with time.

18. The method of claim 17, wherein the frequency of administration of the medicament or antiangiogenesis compound in combination with i-MP treatment required for therapeutic efficacy is from 12 to 18 weeks in a first year of treatment, from 20-28 weeks in a second year of treatment, from 36 weeks to 1 year in a third year of treatment and as needed in a fourth and subsequent years of treatment.

19. The method of claim 17, wherein the frequency of administration of the medicament or antiangiogenesis compound in combination with i-MP treatment required for therapeutic efficacy is initially the same as treatment in the absence of i-MP treatment for 1 to 6 months.

20. A method of treating CNV secondary to wet AMD, Angioid Streaks, Pathologic Myopia, Central Serous Retinopathy, or other choroidal diseases resulting from inflammatory conditions and idiopathic causes comprising:
administering a medicament in combination with i-MP treatment, wherein the i-MP treatment comprises a computer-implemented i-MP treatment.

21. The method of claim 20, wherein the medicament is selected from the group consisting of antiangiogenesis compounds, anti-inflammatory compound, cytotoxic compounds, immunomodulator compounds and anti-proliferative compounds.

22. The method of claim 21 wherein the medicament is an antiangiogenesis or an anti-inflammatory compound.

23. The method according to claim 22, wherein the medicament comprises an anti-VEGF compound.

24. The method of claim 23, wherein the anti-VEGF compound comprises an antibody or antibody fragment.

25. The method of claim 24, wherein the antibody or antibody fragment comprises bevacizumab.

26. The method of claim 24, wherein the antibody or antibody fragment comprises ranibizumab.

27. The method of claim 24, wherein the anti-VEGF compound comprises an aptamer.

28. The method of claim 27, wherein the aptamer comprises pegaptanib sodium.

29. The method of claim 24, wherein the medicament decreases extracellular protease expression and/or inhibits endothelial cell migration.

30. The method of claim 29, wherein the medicament comprises a steroid-based compound.

31. The method of claim 30, wherein the steroid-based compound comprises anecortave acetate.

32. The method of claim 24, wherein the medicament is vatalanib.

33. The method of claim 22, wherein the medicament comprises triamcinolone or triamcinolone acetonide.

34. The method of claim 20, wherein the medicament is administered up to three weeks after the i-MP treatment.

35. The method of claim 34, wherein the medicament is administered immediately after the i-MP treatment.

36. The method of claim 34, wherein the medicament is administered up to 1 week after i-MP treatment.

37. The method of claim 34, wherein the medicament is administered up to 2 weeks after i-MP treatment.

38. The method of claim 34, wherein the frequency of administration of the medicament in combination with the i-MP treatment required for therapeutic efficacy reduces with time.

39. The method of claim 38, wherein the frequency of administration of the medicament in combination with the i-MP treatment required for therapeutic efficacy is from 12 to 18 weeks in a first year of treatment, from 20-28 weeks in a second year of treatment, from 36 weeks to 1 year in a third year of treatment and as needed in a fourth and subsequent years of treatment.

40. The method of claim 38, wherein the frequency of administration of the medicament or antiangiogenesis compound in combination with the i-MP treatment required for therapeutic efficacy is initially the same as treatment in the absence of the i-MP treatment for 1 to 6 months.

41. The method according to claim 20, wherein the computer-implemented i-MP treatment comprises:
a control method comprising the following steps:
determining and/or inputting at least one dosage parameter and/or at least one application parameter of the therapeutic procedure dependent on patient-related data;
providing instructions to an operator to administer ICG into the patient in accordance with the at least one dosage parameter; and
providing instructions to the operator to apply an output of an application device to a treatment area of the patient in accordance with the at least one application parameter;
wherein at least one of the steps is a computer-implemented step.

42. The method according to claim 41 wherein all of the steps are computer-implemented steps.

43. A method according to claim 41 wherein at least one of the steps is a manually-implemented step.

44. The method according to claim 20, wherein the one or more of the steps of the i-MP treatment are manual controlled, substantially manually controlled, partially computer implemented controlled, substantially computer implemented controlled or combinations thereof.

45. The method according to claim 20, wherein at least two of the steps are manually-implemented steps.

46. The method according to claim 20, wherein at least three of the steps are manually-implemented steps.

47. The method according to claim 20, wherein all of the steps are manually-implemented steps.

48. The method according to claim 20, wherein substantially all of the steps are manually-implemented steps.
